(19) United States
(12) Patent Application Publication
(10) Pub. No.: US 2023/0083754 A1
(43) Pub. Date: Mar. 16, 2023

(54) PROBIOTICS SEPARATED FROM RABBIT FECES AND FOOD CONTAINING PROBIOTICS

(71) Applicant: AGENT Innovation Co., Ltd., Taichung (TW)

(72) Inventors: JYH HORNG SHYU, Taichung (TW); LI YU CHIANG, Taichung (TW); YU HSIN CHANG, Taichung (TW); CHING YU CHIU, Taichung (TW); PEI-JU WANG, Taichung (TW)

(21) Appl. No.: 17/895,071

(22) Filed: Aug. 25, 2022

Related U.S. Application Data
(60) Provisional application No. 63/237,005, filed on Aug. 25, 2021.

(51) Int. Cl. A61K 35/747; C12N 1/20; A23K 10/18; A23K 20/147; A23K 20/163; A23K 50/00

(52) U.S. Cl. CPC A61K 35/747; C12N 1/205; A23K 10/18; A23K 20/147; A23K 20/163; A23K 50/00; A61K 2035/115

(57) ABSTRACT

The present disclosure provides a probiotics of PTA22 from rabbits, a nutritional composition for preparing food of rabbits and a composition for rabbits to degrade oxalic acid. Through this disclosure, the health of rabbits can be ensured and the resistance to pathogenic bacteria can be improved after rabbits consume the food containing PTA22. As well, the probiotic PTA22 can help rabbits reduce the risk of hypercalciuria and calculus.

### CROSS-REFFERENCE TO RELATED APPLICATION

This application claims the priority from U.S. provisional Patent Application No. 63/237,005, filed on Aug. 25, 2021, and the contents of which are hereby incorporated by reference in their entirety for all purposes.

### REFERENCE TO SEQUENCE LISTING

The Sequence Listing XML file submitted via the USPTO Patent Center, with a file name of “Sequence listing.XML”, a creation date of Aug. 25, 2022, and a size of 11 KB, is part of the specification and is incorporated in its entirety by reference herein.

### BACKGROUND

### 1. Field of the Invention

The present disclosure relates to probiotics, in particular to, the present disclosure relates to a probiotic from rabbits and food containing the probiotic for rabbits.

### 2. Description of the Related Art

Rabbits are herbivores fermented in the cecum. The cecum contains a lot of microorganisms and probiotics that help break down the thick cell walls of plants. Then, the incompletely digested chyme is fermented and converted into nutrients that can be absorbed. Usually, foods with low crude fiber content contain higher amounts of carbohydrates, which not only ferment easily and cause abdominal flatulence in rabbits but also promote the abnormal growth of certain bacteria, such as Escherichia coli (E. coli) and Clostridium spp. Moreover, abnormal bacteria growth may cause diarrhea, enterotoxemia, intestinal obstruction, chronic intermittent diarrhea, and other intestinal symptoms.

Thus, in order to enable rabbits to digest various foods better and ensure the health of the gastrointestinal tracts of rabbits, the present disclosure provides a grass cake containing probiotics. Rabbits can ingest appropriate probiotics when eating the grass cake so as to prevent gastrointestinal disorders and other diseases.

### SUMMARY

In light of the foregoing, this disclosure provides novel probiotics separated from rabbit feces. Besides, these novel probiotics can have higher opportunity to survive in the gastrointestinal tract of rabbits. The ability of acid tolerance, colonization in the gastrointestinal tract and so on of one of the novel probiotics, Lactiplantibacillus Plantarum (denoted as probiotic PTA22 below) is more significant. The 16S rRNA gene sequence of the probiotic PTA22 is SEQ ID No: 3, and the deposited number is BP-03477 in NITE Patent Microorganisms Depositary (NPMD). Moreover, the probiotic PTA22 has oxalic acid degradation activity. Also, the probiotic PTA22 has oxalic acid degradation activity, carboxymethyl cellulose digestion activity, pectinase digestion activity, xylanase digestion activity, and protease digestion activity, and wherein the probiotic PTA22 can inhibit the growth of a pathogenic bacterium comprising at least one of Bacillus Cereus, Staphylococcus Aureus, Klebsiella Pneumoniae, and Salmonella Enterica, Shigella Sonnei, Streptococcus Pneumoniae, Pseudomonas Aeruginosa, and E. coli (ETEC). The probiotic PTA22 is resistant to an antibiotic comprising at least one of Aminoglycosides antibiotics, Sulfonamide antibiotics, Quinolone antibiotics, and the derivatives thereof.

In one aspect, an embodiment of this disclosure provides a nutritional composition for preparing food of rabbits. The nutritional composition for preparing food of rabbits comprises a probiotic mixture containing the probiotic PTA22, a postbiotic thereof or a combination thereof, a biological material with high biological value protein, an oligosaccharide and an excipient.

In another aspect, an embodiment of this disclosure provides a composition for rabbits to degrade oxalic acid. The composition for rabbits comprises the probiotic PTA22 in an effective amount, a component comprises a biological material with high biological value protein, an oligosaccharide and an excipient.

In short, the embodiments of this disclosure can provide rabbits with the probiotic PTA22 while rabbits eating. In this way, the health of rabbits can be ensured and the resistance to pathogenic bacteria can be improved. As well, the probiotic PTA22 can help rabbits reduce the risk of hypercalciuria and calculus.

### BRIEF DESCRIPTION OF THE DRAWINGS

FIG. 1 shows the test results of Colonies LP1, LP5, LP19, PTA22, PAL44, and SL45 on the blood agar;

FIGS. 2A-2E show the acid tolerance test results of Colonies LP1, LP2, LP5, LP19, PTA22, PAL44, and SL45 at pH 3.0, 2.5, 2.0, 1.5, and 1.0, respectively;

FIGS. 3A-3B show the bile salt tolerance test results of Colonies LP1, LP2, LP5, LP19, PTA22, PAL44, and SL45, respectively;

FIG. 4 shows the results of the hydrophobicity test of Colonies LP5, LP19, PTA22, LP1, and LP2, respectively;

FIG. 5 shows the results of the auto-aggregation test of Colonies LP1, LP2, LP5, LP19, PTA22, PAL44, and SL45, respectively;

FIGS. 6A-6E shows the results of co-aggregation test of Colonies LP1, LP2, LP5, LP19, and PTA22, respectively;

FIG. 7 shows an oxalic acid degradation activity of PTA22 without Mn²⁺ and with Mn²⁺;

FIG. 8A shows the acid tolerance test results of PTA22 at pH 3.0, 2.0, 1.0, 0.5% bile salt and 1.0% bile salt, respectively;

FIG. 8B shows viable bacterial counts of PTA22 under different pH values at 1, 10, 20, 30, 60, 180, and 360 minutes, respectively;

FIG. 9 shows the results of PTA freezing-dried survival rate of PTA22 mixed with various lyoprotectants;

FIGS. 10A-10C show the acid tolerance test results of the freeze-dried bacterial powder at pH 3.0, 2.0, and 1.0, respectively;

FIGS. 11A-11B show the bile salt tolerance test results of the freeze-dried bacterial powder 0.5% bile salt and 1.0% bile salt, respectively;

FIG. 12 shows the comparison diagram of PTA22 mixed with single lyoprotectants and various compositions;

FIGS. 13A-13C show the acid tolerance test results of the composition containing PTA22 at pH 3.0, 2.0, and 1.0, respectively;

FIGS. 14A-14B show the bile salt tolerance test results of the composition containing PTA22 with 0.5% bile salt and 1.0% bile salt, respectively;

FIGS. 15A-15B show the acid tolerance test and bile salt tolerance results of a PBS control and the composition, respectively;

FIG. 15C shows the heat tolerance test results of the PBS control and the composition; and

FIGS. 16A-16B show the grass cakes without PTA22 and the grass cakes with PTA22, respectively.

### DESCRIPTION OF THE PREFERRED EMBODIMENTS

To make the objectives, technical solutions and advantages of the present invention clearer, the technical solutions in the embodiments of the present invention will be described clearly and completely with reference to the accompanying drawings of the present invention. Obviously, the described embodiments are part of, but not all of, the embodiments of the present invention. Based on the embodiments of the present invention, all other embodiments obtained by those skilled in the art without creative work shall fall within the protection scope of the present invention. Unless otherwise defined, the technical or scientific terms used herein shall have the usual meanings understood by those skilled in the art related to the present invention. As used herein, “comprising” and other similar terms mean that the elements or objects appearing before the term encompass the elements or objects listed after the term and their equivalents, without excluding other elements or objects.

### Bacterial Screening and Hemolysis Test

To ensure screened bacteria have no possibility of causing disease, the screened bacteria are tested by hemolysis to confirm whether they cause disease. If colonies are small and gray-white, and there is no hemolysis ring around the colonies, it means that the bacteria have no pathogenicity.

Probiotic strains were cultured by the following method. Firstly, 0.1 g of fecal pellets per rabbit were sampled and then dissolved in MRS broth and Fastidious Anaerobe Broth (FAB), respectively, to form two sample solutions per rabbit. MRS broth was used for culturing aerobes, such as Lactobacillus, and FAB was used for culturing anaerobes. Next, the two sample solutions from each rabbit were centrifuged at 500 rpm, and 100 μL of the supernatants each was serially diluted to form bacterial solutions at a concentration of 10⁻⁷, 10⁻⁸, and 10⁻⁹, relative to the original concentration of the bacterial solutions. Feces from cecum of rabbits were similarly treated, but the serially diluted concentrations of the bacterial solutions were 10⁻⁸, 10⁻⁹, and 10⁻¹⁰, relative to the original concentration of the bacterial solutions.

Subsequently, the bacterial solutions with various concentrations obtained from MRS broth and FAB were evenly plated on MRS agar or Fastidious Anaerobe Agar (FAA), respectively, to confirm the hemolytic property. The plates of MRS agar were used to culture aerobes, such as Lactobacillus, under aerobic conditions under 37° C. for 12 hrs. The plates of FAA were used to culture anaerobes under both aerobic and anaerobic conditions under 37° C. for 12 hrs.

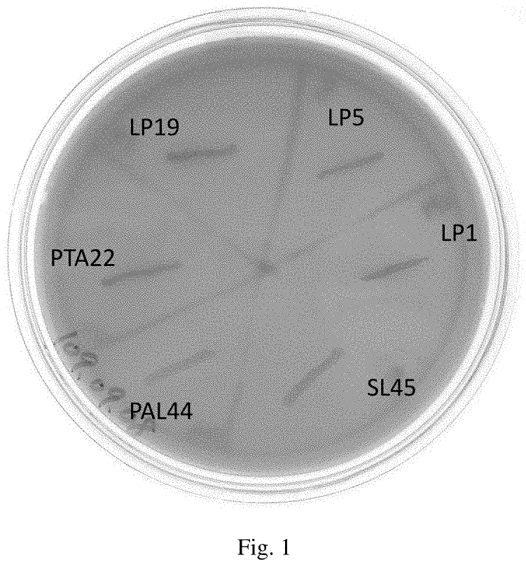

Then, the hemolytic property of every grown single colony was confirmed by plating on a blood agar and then numbered. FIG. 1 shows the test results of Colonies LP1, LP5, LP19, PTA22, PAL44, and SL45 on the blood agar. Colonies LP1 and LP2 (not shown in FIG. 1) are commercially available human intestinal probiotics, which are used as positive control 1 and control 2 for other strains, and in subsequent experiments the same. Colonies LP5, LP19, PTA22, PAL44, and SL45 were obtained from rabbits. In FIG. 1, Colonies LP1, LP5, LP19, PTA22, PAL44, and SL45 are non-hemolytic.

Next, the non-hemolytic colonies from the blood agar were cultured in 3 mL MRS broth or FAB. After cultured, the mediums (MRS broth or FAB) were aliquoted into microcentrifuge tubes with 1 mL×3, respectively. Next, 250 μL glycerol was added into two of the three microcentrifuge tubes for preparing frozen tubes for storing, and the rest of the microcentrifuge tubes was configured to extract DNA and DNA sequencing.

Further, the bacteria of Colonies LP5, LP19, PTA22, PAL44, and SL45 are identified by sequencing 16S rRNA genes, and the sequencing protocols are as follow.

(1) DNA Extraction: DNA was extracted by CTAB (cetyl trimethylammonium bromide) liquid nitrogen frozen method. Then, DNA and protein were separated by 24:1 chloroform/isoamyl alcohol. Next, DNA was precipitated by EtOH and re-dissolved by elution buffer. Finally, the correctness of DNA was checked with 1% agarose gel electrophoresis.

(2) PCR & Gel Purification: 16S rRNA genes of the bacteria were amplified by PCR. The 16S rRNA genes of the bacteria were amplified by DNA polymerase and 16S primer (Forward/Reverse). The base-pair sizes of the 16S rRNA genes of the bacteria were verified by 1% agarose gel electrophoresis. Then, the target 16S rRNA genes of bacteria were purified by Favorgen FavorPrep™ GEL/PCR purification kit.

(3) Ligation & Transformation: T&A™ vector, containing Ampr gene, and 16S rRNA genes were ligated by T4 DNA ligase. Next, the ligated vector was transformed into DH5a cells of E. Coli. Then, the transformed E. Coli was plated on LB agar containing Ampicillin, which was used to select DH5a cells that are successfully transformed by T&A™ vector. After overnight, colonies on the LB agar were selected to amplify for the next step.

(4) Plasmid Extraction & Enzyme Digestion: Plasmids of the selected transformed colonies of DH5a cells were extracted by Favorgen FavorPrep™ plasmid extraction kit. Then, the extracted plasmids were cut by EcoRI or HindIII restriction enzyme to confirm whether the 16S rRNA genes are correctly ligated. Next, the base-pair sizes of the products from the restriction enzymes above were checked by 1% agarose gel electrophoresis to compare with the PCR results to check whether the 16S rRNA genes had successfully inserted into the T&A™ vector or not.

(5) Sequencing: The plasmids of which the restriction enzyme's cutting sites had been identified were sequenced, and the sequencing results were then analyzed. Please refer to the Sequence Table for the sequence of the bacteria of Colonies LP5, LP19, PTA22, PAL44, and SL45.

### Acid Tolerance Test

Since the gastric acid of rabbits is only active when the pH value is about 1.5-1.0, an acid tolerance of the bacteria has to be tested. The acid tolerance test was performed to test how long the bacteria of Colonies LP5, LP19, PTA22, PAL44, and SL45 each can survive in the gastric acid environment.

Firstly, the pH of MRS broth was adjusted to 1.0, 1.5, 2.0, 2.5, or 3.0 with HCl. Next, 100 μL of each bacterial solution of the bacteria, LP5, LP19, PTA22, PAL44, and SL45 was taken, and was respectively added into the following solutions of MRS broth: negative control (only MRS broth), pH 1.0, pH 1.5, pH 2.0, pH 2.5, and pH 3.0. Then, the bacterial solutions were cultured at 37° C. for 4 hours, and OD₆₀₀ of these bacterial solutions was measured per hour.

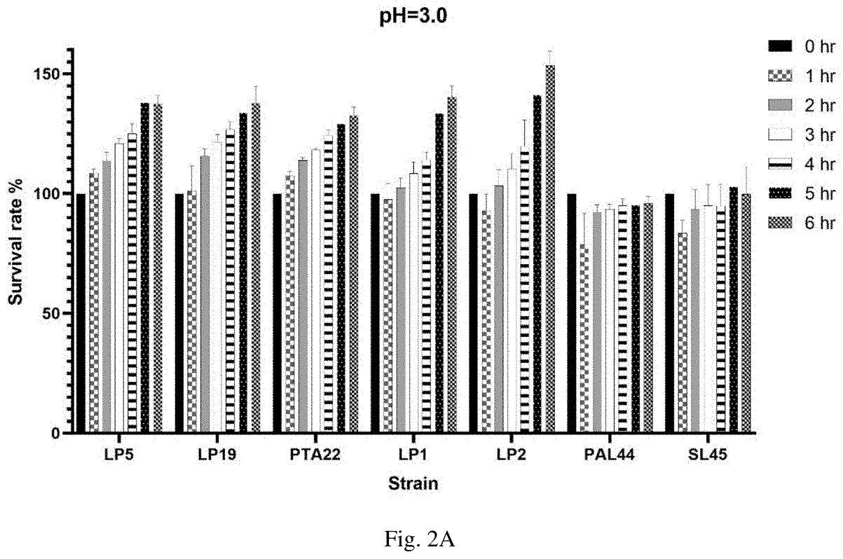

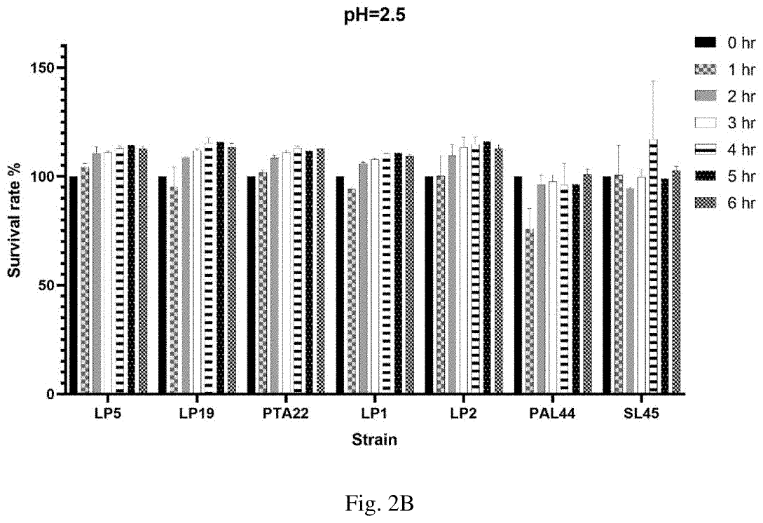

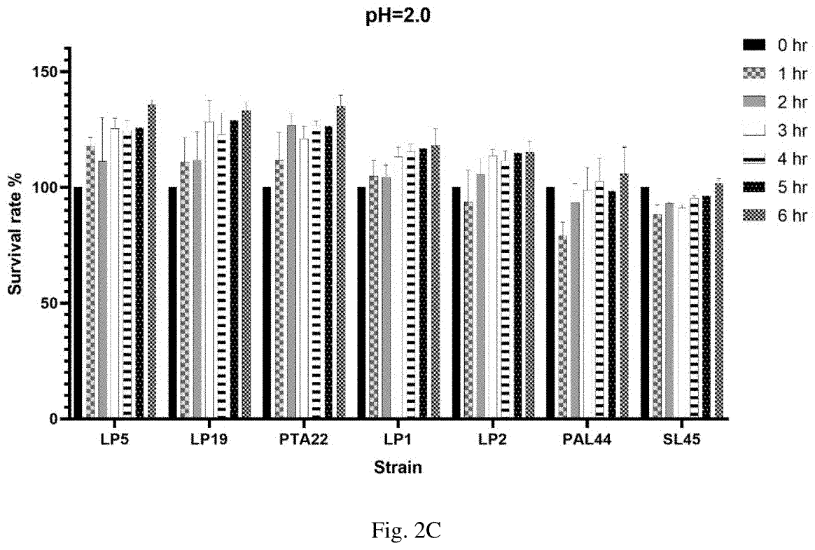

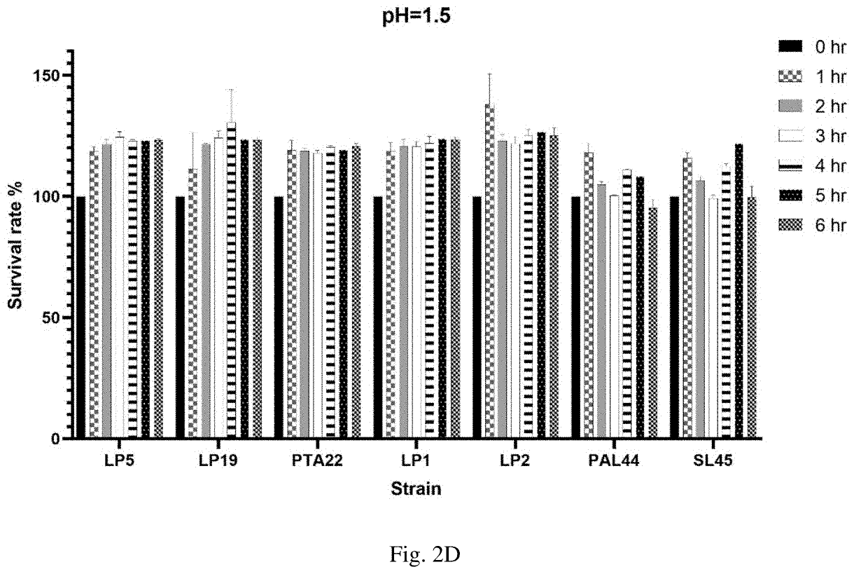

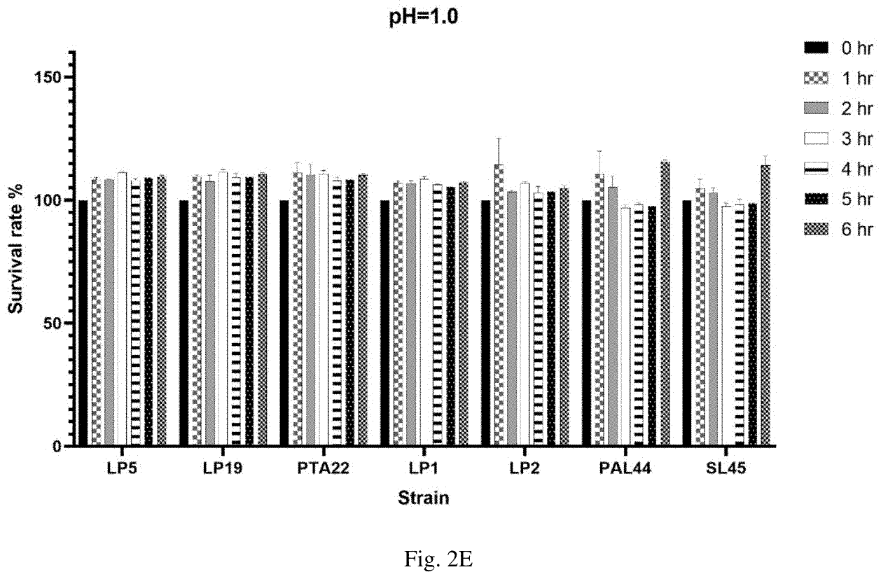

FIGS. 2A-2E show the acid tolerance test results of Colonies LP1, LP2, LP5, LP19, PTA22, PAL44, and SL45 at pH 3.0, 2.5, 2.0, 1.5, and 1.0, respectively. In this acid tolerance test, the test time was 0-6 hours, as described in previous research [Susan M. Smith (2012). Gastrointestinal Physiology and Nutrition of Rabbits (pages 162-173). WB Saunders]. In FIGS. 2A-2E, the survival rate of Colonies LP5, LP19 and PTA22 shows that Lactiplantibacillus plantarum found from rabbits can withstand and even thrive in the acidic environments within a certain period. Besides, in 1 to 2 hours, the acid-tolerance survival rate of PTA22 is the best.

### Bile Salt Tolerance Test

Similar to the acid tolerance test, the bile salt tolerance test was performed to test the bacterial activity of Colonies LP5, LP19, PTA22, PAL44, and SL45 each in the intestinal environment.

Firstly, solutions of MRS broth containing 0.5 wt % and 1.0 wt % bile salt were prepared. Next, 100 μL of each bacterial solution of the bacteria of Colonies, LP5, LP19, PTA22, PAL44, and SL45 was taken, and was respectively added into the following solutions of MRS broth: negative control (MRS broth only), 0.5 wt % bile salt, and 1.0 wt % bile salt. Then, the bacterial solutions were cultured at 37° C. for 8 hours, and OD600 of these bacterial solutions was measured per hour.

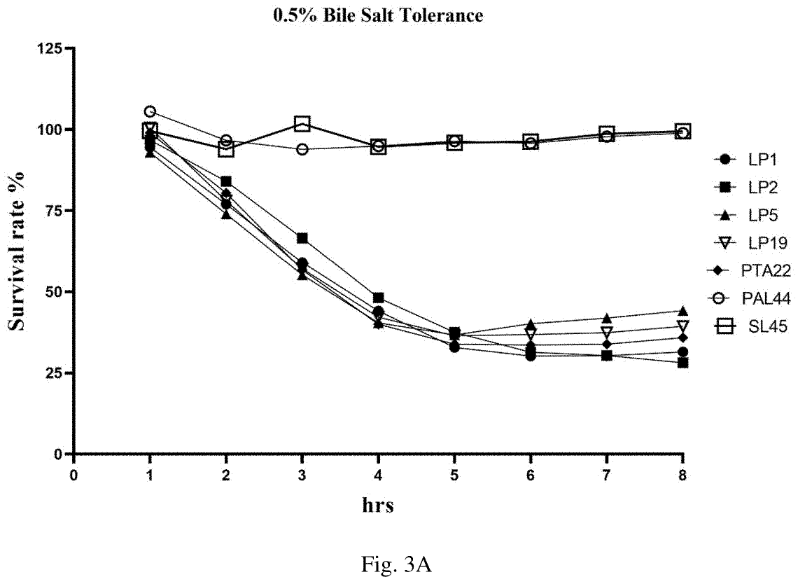

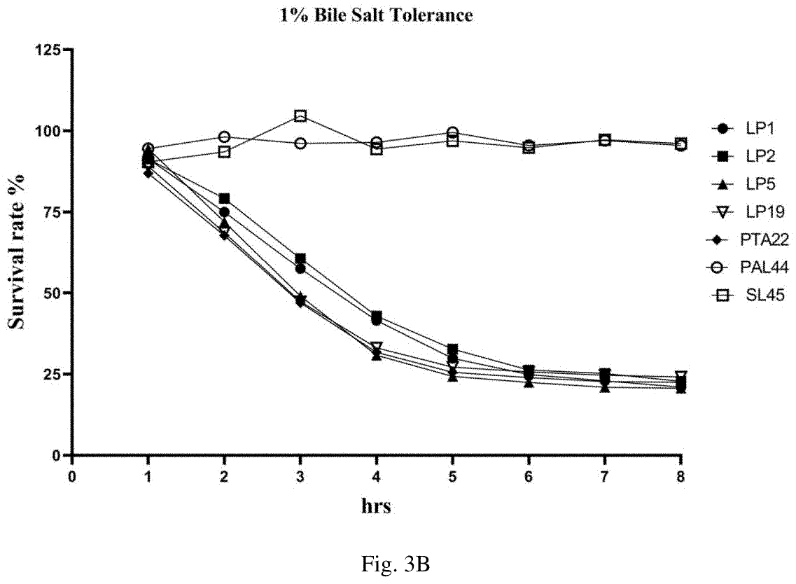

FIGS. 3A-3B show the bile salt tolerance test results of Colonies LP1 (positive control 1), LP2 (positive control 2), LP5, LP19, PTA22, PAL44, and SL45, respectively. From FIGS. 3A-3B, it can be known that the bacteria of Colonies PAL44 and SL45 had better activity and could stably grow in the intestine. As for Colonies LP5, LP19, and PTA 22 of Lactiplantibacillus Plantarum, no obvious difference was observed. According to Gastrointestinal Physiology and Nutrition of Rabbits (Susan M. Smith, 2012, pages 162-173), the times for foods passing through the duodenum and ileum are 10-20 minutes and 30-60 minutes, respectively. In FIGS. 3A-3B, the survival rate of the bacteria LP5, LP19, PTA22, PAL44, and SL45 in 0.5% or 1% bile salt environment was declined over time. Therefore, in product development, a protective agent is very important for the viable amount of the bacteria.

### Analysis of Degrading Enzyme Activities

In order to explore the ability of the bacteria, LP5, LP19, PTA22, PAL44, and SL45 to degrade cellulose, an analysis of degrading enzyme activity was performed. The degrading enzymes included carboxymethyl cellulase (CMCase), xylanase, amylase, pectinase, and protease.

Before entering the activity test, the reagents used in this experiment, Congo red and Iodine reagent, are introduced first. For the activity tests of carboxymethyl cellulase (CMCase), xylanase, and pectinase, Congo red was used in the stain method. Congo red is able to synthesize red complexes with cellulose, but does not react with the products after cellulose hydrolysis. Thus, if there is a cellulolytic bacterium, when Congo red is added, a transparent ring around the colony appears. This means that the bacteria break down cellulose, so that the cellulose cannot synthesize red complexes with Congo red. For the activity test of amylase, starch reacting with Iodine reagent produces a purple complex. If starch is degraded, a transparent ring around the colony appears.

For the activity tests of carboxymethyl cellulase (CMCase), xylanase, and pectinase, the MRS agars above were stained by 0.1 wt % of Congo red aqueous solution for 30 minutes and then destained by 1 M NaCl aqueous solution. For the activity test of amylase, the MRS agar containing 0.02 wt % starch was stained by 1 wt % Iodine reagent for 1 minute and then destained by ddH₂O. Besides, for the activity test of protease, there is no stain used and was visually observed. The experimental results of the activity test of protease are also interpreted through whether a transparent ring around the colony appears or not.

The analysis of degrading enzyme activity was qualitative and quantitative. In the qualitative analysis of degrading enzyme activity, the bacteria were plated on a MRS agar by 4-zones streaking method and cultured with 37° C. for 2 days. Next, a single colony on the 4-zone MRS agar was taken and then cultured on MRS agar containing 1 wt % carboxymethyl cellulose, 0.05 w t% xylan, 1 wt % pectin, 0.02 wt % starch, or 1 wt % skim milk for protease for 1 day.

The test results of the qualitative analysis are shown in Table 1 below. From the test results, it can be known that LP1, LP2, LP5, LP19, and PTA 44 all have the activity of CMCase, xylanase, pectinase, and protease since they are all belong to the same genus of Lactiplantibacillus Plantarum.

TABLE 1
Qualitative analysis results of carboxymethyl cellulase
(CMCase), xylanase, amylase, pectinase, and protease.

| Colony | CMCase | xylanase | pectinase | amylase | protease |
|---|---|---|---|---|---|
| LP1 | + | + | + | − | + |
| LP2 | + | + | + | − | + |
| LP5 | + | + | + | − | + |
| LP19 | + | + | + | − | + |
| PTA22 | + | + | + | − | + |
| PAL44 | − | − | − | + | − |
| SL45 | − | − | − | + | − |

In the quantitative analysis of degrading enzyme activity, the concentrations of bacterial solutions were taken for the analysis when the OD₆₀₀=1.0. Oxford cups were used to confine the bacterial growth area located on MRS agars. After culturing with 37° C. for 2 days, the activities of the degrading enzymes were observed by stain method. As for the activity test of protease, the result may be directly observed by eyes and thus no dye was needed. The data above were summarized in Table 2 below.

TABLE 2
The substrates and dyes used in the activity test of carboxymethyl
cellulase (CMCase), xylanase, amylase, pectinase, and protease.

| Degrading | Substrate in | Dye used in |
|---|---|---|
| Enzyme | MRS Agars | Stain Method |
| carboxymethyl | 1 wt % carboxymethyl | 0.1 wt % Congo red (aq) |
| cellulase | cellulose | |
| (CMCase) | | |
| xylanase | 0.05 wt % xylan | 0.1 wt % Congo red (aq) |
| pectinase | 1 wt % pectin | 0.1 wt % Congo red (aq) |
| amylase | 0.02 wt % starch | 1 wt % Iodine solution (aq) |
| protease | 1 wt % skim milk | None |

The test results were summarized in Table 3 below. From Table 3, it can be known that the activity of xylanase and pectinase of Colonies LP5, LP19, and PTA22 from rabbits were better than Colonies LP1 and LP2. In addition, the activities of CMCase and protease of Colonies LP5, LP19, and PTA22 are comparable with Colonies LP1 and LP2.

TABLE 3
The diameters (mm) of microbial decomposition zones for
Colonies LP1, LP2, LP5, LP19, PTA22, PAL44, and SL45

| Colony | CMCase | xylanase | pectinase | amylase | protease |
|---|---|---|---|---|---|
| LP1 | 13 | 11 | 12 | — | 8 |
| (Positive | | | | | |
| control 1) | | | | | |
| LP2 | 13 | 12 | 13 | — | 9 |
| (Positive | | | | | |
| control 2) | | | | | |
| LP5 | 11 | 21 | 16 | — | 8 |
| LP19 | 14 | 21 | 18 | — | 9 |
| PTA22 | 11 | 20 | 19 | — | 9 |
| PAL44 | — | — | — | 11 | — |
| SL45 | — | — | — | 13 | — |

### Antibacterial Activity Analysis—Agar Diffusion Method

Dysbiosis of intestinal flora may cause various diseases. The intake of probiotics in rabbits is very important to inhibit the proliferation of pathogenic bacteria, regulate the balance of flora and improve immunity. Thus, the bacteria of Colonies LP1, LP2, LP5, LP19, PTA22, PAL44, and SL45 were used to test the inhibitory activity to some pathogenic bacteria, such as Salmonella Enterica, Shigella Sonnei, Klebsiella Pneumoniae, Streptococcus Pneumoniae, Bacillus Cereus, Staphylococcus Aureus, and Pseudomonas Aeruginosa. The details of this method are described below.

Agar Diffusion Method-1: Use the Bacteria for Testing

To test the growth inhibition of the bacteria against pathogenic bacteria, the experiment was designed as follow. After cultured in MRS broth/FAB with 37° C. for 12 hours, 100 μL bacterial solutions were evenly plated on MRS agar/FAA and then dried for 10 minutes, respectively. Next, tips were used to form several holes in the MRS agar/FAA. Subsequently, 100 μL bacterial solutions of the pathogenic bacteria described above were added into the holes and then cultured at 37° C. for 12 hours. The diameters of pathogenic bacteria growth zone on FAA plated by bacterial solutions of Colonies LP1, LP2, LP5, LP19, PTA22, PAL44, and SL45, including a negative control group (no bacteria solution), are listed in Table 4 below.

This experiment is designed to test whether the Colonies on the FAA are able to inhibit the growth of the pathogenic bacteria in the holes. Therefore, the smaller growth zone indicates the better inhibitory effect of the bacteria. To clarify the difference in the growth zone diameters, the difference in the growth zone diameters between the experimental groups and control groups thereof are listed in Table 5 below. From Table 5, it can be clearly seen that colonies LP5, LP19, PTA22, PAL44 and SL45 all had growth-inhibiting effects on pathogenic bacteria, especially Bacillus Cereus, Staphylococcus Aureus, Klebsiella Pneumoniae, and Salmonella Enterica.

TABLE 4
Diameters (mm) of pathogenic bacteria growth zones on FAA plated by bacterial solutions of
Colonies LP1, LP2, LP5, LP19, PTA22, PAL44, and SL45, including a control group thereof.

| | *FAA | LP1 | LP2 | | | | | |
|---|---|---|---|---|---|---|---|---|
| | (negative | (positive | (positive | | | | | |
| FAA Growth zone (mm) | control) | control 1) | control 2) | LP5 | LP19 | PTA22 | PAL44 | SL45 |
| Salmonella Enterica | 13 | 9.5 | 9.5 | 9.5 | 11 | 10 | 10 | 9 |
| Shigella Sonnei | 11.5 | 9.5 | 9.5 | 9.5 | 11 | 10 | 9 | 9.5 |
| Klebsiella Pneumoniae | 15 | 11 | 11 | 11 | 12 | 13 | 10 | 10 |
| Streptococcus Pneumoniae | 11.5 | 9.5 | 9.5 | 9.5 | 9.5 | 9 | 9 | 9 |
| Bacillus Cereus | 22 | 12 | 13 | 12 | 18 | 16 | 11 | 11 |
| Staphylococcus Aureus | 13 | 10 | 9 | 9 | 10 | 9 | 10 | 9.5 |
| Pseudomonas Aeruginosa | 12 | 11 | 11.5 | 11 | 11.5 | 11.5 | 10 | 10 |

*FAA control: a negative control group, pathogenic bacteria only, none of Colonies LP1, LP2, LP5, LP19, PTA22, PAL44, and SL45 were plated on FAA.

TABLE 5
Difference in the growth zone diameters between the
experimental groups and control groups thereof.

| | LP1 | LP2 | | | | | |
|---|---|---|---|---|---|---|---|
| | (positive | (positive | | | | | |
| Diameters difference (mm) | control 1) | control 2) | LP5 | LP19 | PTA22 | PAL44 | SL45 |
| Salmonella Enterica | −3.5 | −3.5 | −3.5 | −2 | −3 | −3.5 | −4 |
| Shigella Sonnei | −2 | −2 | −2 | −0.5 | −1.5 | −2.5 | −2 |
| Klebsiella Pneumoniae | −4 | −4 | −4 | −3 | −2 | −5 | −5 |
| Streptococcus Pneumoniae | −2 | −2 | −2 | −2 | −2.5 | −2.5 | −2.5 |
| Bacillus Cereus | −10 | −9 | −10 | −4 | −6 | −11 | −11 |
| Staphylococcus Aureus | −3 | −4 | −4 | −3 | −4 | −3 | −3.5 |
| Pseudomonas Aeruginosa | −1 | −0.5 | −1 | −0.5 | −0.5 | −2 | −2 |

### Agar Diffusion Method-2: Use the Cell-Free Solution (CFS) for Testing

Firstly, the CFSs (containing metabolites) were prepared. Besides, when probiotics break down fiber and convert the fiber into metabolites, the metabolites are postbiotics. The supernatants of the cultured bacteria were collected by centrifuged at 5000 rpm, after the colonies LP1 (the positive control 1), LP2 (the positive control 2), LP5, LP19, PTA22, PAL44, and SL45 were cultured in MRS broth/FAB at 37° C. for 48 hours.

Next, the pathogenic bacteria described above were also cultured with 37° C. until the concentration thereof reached 1×10⁸ CFU/mL, respectively. 100 μL bacterial solutions of the pathogenic bacteria described above were evenly plated on Tryptone Soy Agar (TSA) and stay for 10 minutes. Then, tips were used to form several holes in the TSA. Subsequently, 100 μL bacterial solutions of the colonies described above were added into the holes and then cultured at 37° C. for 12 hours.

The measured diameters of inhibitory zones of the cell-free solutions of Colonies LP1, LP2, LP5, LP19, PTA22, PAL44, and SL45, including a control group thereof, are listed in Table 6 below. This experiment is designed that the CFSs in the holes prevent the pathogenic bacteria on the plates from growing. Therefore, the larger zone indicates the better antibacterial effect. In Table 6, the cell-free solutions of Colonies LP1, LP2, LP5, LP19 and PTA22 exhibited significant inhibitory activities.

TABLE 6
Diameters of inhibitory zones of the cell-free solutions of Colonies LP1, LP2,
LP5, LP19, PTA22, PAL44, and SL45, including a negative control group thereof.

| Inhibitory zone (mm) | *ddH₂O | *AP | *KM | LP1 | LP2 | LP5 | LP19 | PTA22 | PAL44 | SL45 |
|---|---|---|---|---|---|---|---|---|---|---|
| Salmonella Enterica | 8 | 8 | 16 | 17 | 16 | 15 | 15 | 17 | 8 | 8 |
| Shigella Sonnei | 8 | 12 | 20 | 17 | 16 | 15 | 15 | 17 | 8 | 8 |
| Klebsiella Pneumoniae | 8 | 8 | 20 | 17 | 18 | 17 | 19 | 19 | 8 | 8 |
| Streptococcus Pneumoniae | 8 | 8 | 20 | 18 | 18 | 17 | 17 | 17 | 8 | 8 |
| Bacillus Cereus | 8 | 8 | 20 | 16 | 14 | 14 | 15 | 16 | 8 | 8 |
| Staphylococcus Aureus | 8 | 19 | 23 | 23 | 18 | 12 | 23 | 24 | 8 | 8 |
| Pseudomonas Aeruginosa | 8 | 8 | 20 | 15 | 15 | 15 | 16 | 15 | 8 | 8 |
| Escherichia coli (ETEC) | 8 | 8 | 17 | 14 | 15 | 15 | 14 | 15 | 8 | 8 |

*ddH₂O: a negative control group, ddH₂O only, none of Colonies LP1, LP2, LP5, LP19, PTA22, PAL44, and SL45 were added in the hole.
*AP: Ampicillin
*KM: Kanamycin

### Antibacterial Activity Analysis—Minimum Inhibitory Concentration & Minimum Bacterial Concentration

Minimum inhibitory concentration (MIC) refers to the minimum concentration at which the development of bacteria can be blocked and antibacterial resistance can be observed after cultured. The lower MIC means the better effect on bacteria.

First, cell-free solutions of Colonies LP1, LP2, LP5, LP19, and PTA22 were respectively prepared. The supernatants of the cultured colonies described above were collected, as cell-free solutions (CFSs), by centrifuge at 5000 rpm after cultured the colonies in a nutrient broth and shaking at 37° C. for 48 hours. Next, each of the CFSs was diluted to concentrations of 256, 128, 64, 32, 16, 8, 4, 2, 1, 0.5, 0.25, 0.125 μL/mL on 96-well plates by the nutrient broth. In addition, nutrient broth containing 256, 128, 64, 32, 16, 8, 4, 2, 1, 0.5, 0.25, 0.125 μg/mL Ampicillin or Kanamycin was also added to 96-well plates to be positive control groups.

Next, 100 μL each of the CFSs and 100 μL bacterial solutions of the pathogenic bacteria were respectively added to each well of a 96-well plate. Thus, the total volume in each well was 200 μL. The tested pathogenic bacteria included Salmonella Enterica, Shigella Sonnei, Klebsiella Pneumoniae, Streptococcus Pneumoniae, Bacillus Cereus, Staphylococcus Aureus, Pseudomonas Aeruginosa, and Escherichia Coli.

As well, 100 μL each of the nutrient broth containing Ampicillin or Kanamycin and 100 μL bacterial solutions of the pathogenic bacteria were respectively added to each well of a 96-well plate. Thus, the total volume in each well was 200 μL.

Then, initial values of OD₆₀₀ in each well were recorded. Subsequently, the 96-well plates were cultured at 37° C. for 24 hours, and the OD₆₀₀ values thereof were re-recorded for determining the MICs of each colony described above.

Finally, 100 μL CFSs of the next three concentrations below MIC were taken to be plated on nutrient agar and then cultured at 37° C. for 48-72 hours. The concentrations of the CFSs that no colonies grown on nutrient agar were determined to be the minimum bacterial concentration (MBCs).

The determined MICs and MBCs of the colonies descried above are respectively listed in Tables 7 and 8 below. In Tables 7 and 8, PTA22 had better inhibitory activity against Bacillus Cereus and Staphylococcus Aureus.

TABLE 7
MICs of Colonies LP1, LP2, LP5, LP19, PTA22, PAL44, and SL45.

| | Enrofloxacin | Ampicillin | Kanamycin | LP1 | LP2 | LP5 | LP19 | PTA22 |
|---|---|---|---|---|---|---|---|---|
| Microorganism | Concentration (μg/mL) | | | Concentration (μL/mL) | | | | |
| Salmonella Enterica | <0.125 | N | 64 | 32 | 32 | 32 | 32 | 32 |
| Shigella Sonnei | <0.125 | 8 | 16 | 32 | 32 | 32 | 32 | 32 |
| Klebsiella Pneumoniae | 32 | N | 32 | 32 | 32 | 32 | 32 | 32 |
| Streptococcus Pneumoniae | 0.25 | N | 8 | 32 | 32 | 32 | 32 | 32 |
| Bacillus Cereus | 8 | N | 256 | 32 | 32 | 32 | 32 | 16 |
| Staphylococcus Aureus | 1 | 0.5 | 4 | 16 | 16 | 32 | 32 | 16 |
| Pseudomonas Aeruginosa | 16 | N | 8 | 32 | 32 | 32 | 32 | 32 |
| Escherichia Coli (ETEC) | 4 | N | 64 | 32 | 32 | 32 | 32 | 32 |

TABLE 8
MBCs of Colonies LP1, LP2, LP5, LP19, PTA22, PAL44, and SL45.

| | Enrofloxacin | Ampicillin | Kanamycin | LP1 | LP2 | LP5 | LP19 | PTA22 |
|---|---|---|---|---|---|---|---|---|
| Microorganism | Concentration (μg/mL) | | | Concentration (μL/mL) | | | | |
| Salmonella Enterica | <0.125 | N | 128 | 32 | 32 | 32 | 32 | 32 |
| Shigella Sonnei | <0.125 | 16 | 64 | 32 | 32 | 64 | 32 | 32 |
| Klebsiella Pneumoniae | 32 | N | 64 | 32 | 32 | 64 | 32 | 64 |
| Streptococcus Pneumoniae | 0.5 | N | 16 | 32 | 32 | 32 | 32 | 32 |
| Bacillus Cereus | 8 | N | >256 | 32 | 64 | 64 | 16 | 32 |
| Staphylococcus Aureus | 1 | 1 | 8 | 32 | 32 | 32 | 32 | 16 |
| Pseudomonas Aeruginosa | 16 | N | 16 | 64 | 32 | 64 | 64 | 64 |
| Escherichia Coli (ETEC) | 8 | N | 128 | 64 | 32 | 32 | 64 | 64 |

### Antibacterial Activity Analysis—Antibiotic Susceptibility Test

To test bacterial susceptibility to antibiotics, such as Aminoglycosides antibiotics, Sulfonamide antibiotics, Quinolone antibiotics, Ampicillin, Cefotaxime, Chloramphenicol, Erythromycin, Rifampicin and Tetracycline, the experimental protocol were as follow. Further, the Quinolone antibiotics comprise Ciprofloxacin, the Aminoglycosides antibiotics comprise Kanamycin and Vancomycin, and the Sulfonamide antibiotics comprise Sulfamethoxazole. The antibiotics above were diluted by nutrient broth to a series of concentrations of 256, 128, 64, 32, 16, 8, 4, 2, 1, 0.5, 0.25, 0.125 μg/mL in wells of 96-well plates, and the volume of the antibiotic solution was 100 μL in each well. Next, 100 μL bacterial solutions of the tested colonies LP1, LP5, LP19, PTA22, PAL44, and SL45 were respectively added into each well to a total volume of 200 μL. The initial absorbance at 600 nm (OD₆₀₀) of each well was recorded. Then, the 96-well plates were placed at 37° C. and cultured for 24 hours. Next, the OD₆₀₀ of each well was measured again to determine minimum inhibitory concentration. The antibiotic susceptibility test results are shown in Table 9 below. In Table 9, some data of other Lactiplantibacillus Plantarum (L. pl 24-2L, L. pl 24-2L, L. plantarum 299, and L. plantarum 299v) from published literatures are also listed.

In Table 9, the minimum inhibitory concentrations of kanamycin, sulfamethoxazole, and vancomycin to the Colonies LP1, LP2, LP5, LP19, and PTA22 were quite high (>256 μg/mL). The minimum inhibitory concentrations of ciprofloxacin to the Colonies LP1, LP2, LP5, LP19, and PTA22 were next high (>128 μg/mL). These results show that Colonies LP1, LP2, LP5, LP19, and PTA22 were resistant to ciprofloxacin, kanamycin, sulfamethoxazole, and vancomycin. Comparing with European Food Safety Authority (EFSA) MIC breakpoints species L. plantarum (listed on the last column of Table 9), Colony PTA22 had higher tolerance to the Chloramphenicol, Kanamycin, and Tetracycline but had higher susceptibility to the Ampicillin.

TABLE 9
Minimum inhibitory concentrations (μg/mL) of colons LP1, LP2, LP5, LP19, and PTA22, as well as some data of other Lactiplantibacillus Plantarum
(L. pl 24-2L, L. pl 24-2L, L. plantarum 299, and L. plantarum 299v) from published literatures.

| | | | | | | | | | | EFSA, MIC |
|---|---|---|---|---|---|---|---|---|---|---|
| | | | | | | L. pl | L. pl | L. plantarum | L. plantarum | breakpoints species |
| Antibiotics | LP1 | LP2 | LP5 | LP19 | PTA22 | 24-2L | 24-5D | 299 | 299v | L. plantarum |
| Ampicillin | 0.125 | 0.25 | 0.25 | 0.25 | 0.5 | 0.38 | 1 | 0.094 | 0.094 | 4 |
| Cefotaxime | <0.125 | <0.125 | <0.125 | <0.125 | <0.125 | N | N | 0.094 | 0.094 | N |
| Chloramphenicol | 8 | 8 | 16 | 16 | 32 | 4 | 6 | 2 | 2 | 8 |
| Ciprofloxacin | 256 | 128 | 128 | 256 | 256 | N | N | N | N | N |
| Erythromycin | 1 | 2 | 2 | 2 | 4 | 0.75 | 0.75 | 0.75 | 1 | 4 |
| Enrofloxacin | 64 | 64 | 64 | 64 | 64 | N | N | N | N | N |
| Kanamycin | >256 | >256 | >256 | >256 | >256 | 32 | 48 | >256 | >256 | 64 |
| Rifampicin | 1 | 1 | 1 | 2 | 2 | N | N | N | N | N |
| Sulfamethoxazole | >256 | >256 | >256 | >256 | >256 | N | N | N | N | N |
| Tetracycline | 32 | 32 | 32 | 32 | 64 | N | N | N | N | 32 |
| Vancomycin | >256 | >256 | >256 | >256 | >256 | >256 | >256 | >256 | >256 | Not required |

1. LP and PTA22: Lactiplantibacillus Plantarum
2. L. pl 24-2 and LL. pl 24-5D (Lactiplantibacillus Plantarum): from Georgieva et al., 2015. (Georgieva, R., Yocheva, L., Tserovska, L., Zhelezova, G., Stefanova, N., Atanasova, A., & Karaivanova, E. (2015). Antimicrobial activity and antibiotic susceptibility of Lactobacillus and Bifidobacterium spp. intended for use as starter and probiotic cultures. Biotechnology & Biotechnological Equipment, 29(1), 84-91.)
3. L. plantarum 299 and L. plantarum 299v (Lactiplantibacillus Plantarum): from Klarin et al., 2019. (Klarin, B., Larsson, A., Molin, G., & Jeppsson, B. (2019). Susceptibility to antibiotics in isolates of Lactobacillus plantarum RAPD-type Lp299v, harvested from antibiotic treated, critically ill patients after administration of probiotics. MicrobiologyOpen, 8(2), e00642.
4. N (Not specified/tested)
5. EFSA: European Food Safety Authority (EFSA (2005). Opinion of the scientific panel on additives and products or substances used in animal feed on the updating of the criteria used in the assessment of bacteria for resistance to antibiotics of human or veterinary importance. The EFSA Journal, 223, 1-12.)

### Adhesion Test—Hydrophobicity

During the colonization of probiotics in the gastrointestinal tract, the first step is the attachment of bacteria to the host cell tissue. Hydrophobicity determines the ability of bacteria to adhere, which is the key to whether probiotics can thrive in the gastrointestinal tract of rabbits. Hence, the hydrophobic experiment of adhesion test was designed as follow.

After culturing bacterial solutions of Colonies LP1, LP2, LP5, LP19, and PTA22 overnight, the bacterial solutions were centrifuge at 5000 g for 15 minutes. Next, sterile PBS (Phosphate Buffered Saline) solution at about 4° C. (low temperature) was used to wash the pellets. The centrifuging step and washing step were repeated again. Then, PBS was used to suspend the pellets to form an initial bacterial solution with OD₆₀₀=1.0 (denoted as H1).

0.6 mL organic solvent was added into 3 mL initial bacterial solution and vortexed for 2 minutes to form a mixed bacterial solution. The bacterial solution was stayed at room temperature to react. Next, after gently removing the liquid in the lower aqueous phase, the OD₆₀₀ value of the upper organic layer (denoted as H2) was measured. The organic solvent above was n-hexadecane, xylene, or toluene. Hence, the hydrophobicity % may be calculated by the formula (1) below.

$$Hydrophobicity = [ \frac{H 1 - H 2}{H 1} ] \times 100 \% \qquad ( 1 )$$

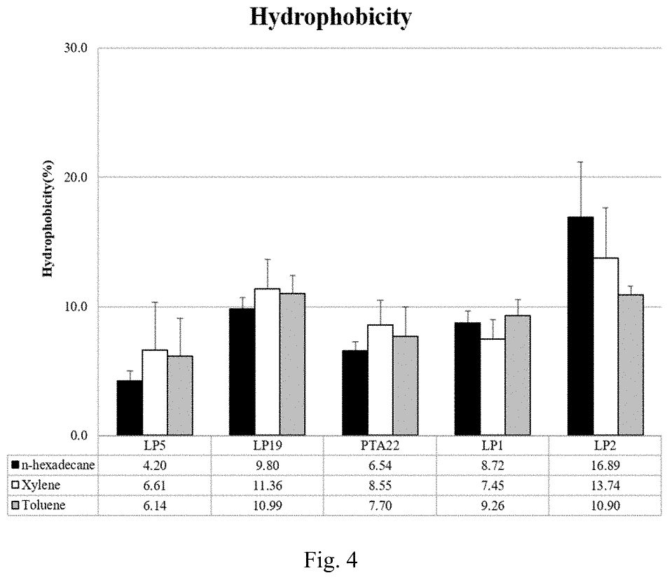

FIG. 4 and Table 10 show the results of the hydrophobicity test. Generally, the hydrophobicity of bacteria is related to the affinity of bacteria to the intestine wall and thus can adhere to the intestine wall well. From FIG. 4, it can be known that, as for LP5, LP19, and PTA 22, only the hydrophobicity of LP19 was higher than 10%. Hence, this result shows that the adherence of LP5, LP19, and PTA 22 to the intestine wall was not good enough.

TABLE 10
Results of the hydrophobicity test

| Organic solvent | LP1 | LP2 | LP5 | LP19 | PTA22 |
|---|---|---|---|---|---|
| n-hexadecane (%) | 8.72 | 16.89 | 4.20 | 9.80 | 6.54 |
| Xylene (%) | 7.45 | 13.74 | 6.61 | 11.36 | 8.55 |
| Toluene (%) | 9.26 | 10.90 | 6.14 | 10.99 | 7.70 |

### Adhesion Test—Auto-Aggregation

In addition to hydrophobicity, the auto-aggregation ability of bacteria has an important impact on bacterial adhesion to intestinal cells. Thus, the next step is to test the auto-aggregation ability of the bacteria, the Colonies LP1, LP2, LP5, LP19, PTA22, PAL44, and SL45.

10 mM PBS solution was prepared. The pH value of the PBS solution was adjusted to pH 7.4, and the PBS solution was then sterilized and standby. A single colony of Colonies LP1, LP2, LP5, LP19, PTA22, PAL44, and SL45 each from MRS agar was taken to be cultured in 3 mL MRS broth at 37° C. with shaking for 16 hours. Next, the bacterial solutions were centrifuged at 6000 rpm for 10 minutes, and the supernatants thereof were removed. The standby PBS solution was used to wash pellets. Then, PBS was used to suspend the pellets to form an initial bacterial solution with OD₆₀₀=0.600 (denoted as A1).

The initial bacterial solution was cultured at 37° C., and OD₆₀₀ values (denoted as A2) thereof were measured after culturing for 1, 3, 6, and 24 hours. Hence, the auto-aggregation may be calculated by the formula (2) below.

$$Auto - aggregation = [ \frac{A 1 - A 2}{A 1} ] \times 100 \% \qquad ( 2 )$$

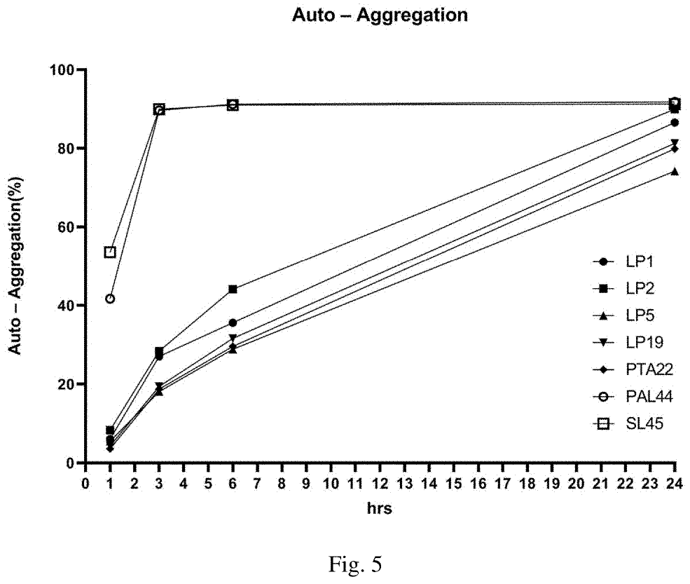

FIG. 5 shows the results of the auto-aggregation test of Colonies LP1, LP2, LP5, LP19, PTA22, PAL44, and SL45, respectively. From FIG. 5, it can be known that PAL44 and SL45 had a much higher auto-aggregation rate and the 90% auto-aggregation was reached at about 3 hours. The 90% auto-aggregation of the rest Colonies PL1, PL2, PL5, PL19, and PTA22 was reached at about 24 hours, and the auto-aggregation rate of these bacteria of colonies PL1, PL2, PL5, PL19, and PTA22 did not show obvious difference. According to Jack C. Leo, et al. (AIMS Microbiol. 2018; 4(1): 140-164.), auto-aggregation is one condition for biofilms formation and one evaluation index for intestinal wall adhering ability. In FIG. 5, the auto-aggregation rate of the colony PTA22 at 24 hours was about 90%. Therefore, after Colony PTA22 passing through the stomach (3-6 hours), the duodenum and ileum (about 1 hour), Colony PTA22 can effectively form biofilms in the cecum and colonize in the cecum.

### Adhesion Test—Co-Aggregation

Co-aggregation eliminates the colonization of gastrointestinal pathogenic bacteria by preventing the pathogenic bacteria from attaching to host tissue. Next, a co-aggregation test is performed to understand the co-aggregation ability of the bacteria, i.e., the Colonies LP1, LP2, LP5, LP19, PTA22, PAL44, and SL45.

A single colony of Colonies LP1, LP2, LP5, LP19, and PTA22 each from MRS agar was taken to be cultured in 3 mL MRS broth at 37° C. with shaking for 16 hours. The bacterial solution of colonies above was mixed with an equal amount of pathogenic bacteria and then vortexed for 30 seconds to form a mixed bacterial solution. Then, the mixed bacterial solution was stayed at 37° C., and OD₆₀₀ values (denoted as Am) thereof were measured after culturing for 1, 3, 6, and 24 hours. Hence, the co-aggregation % may be calculated by the formula (3) below. In formula (3), in addition to Am above, Al is the OD₆₀₀ value of the bacterial solution of the colonies above, and A2 is the OD₆₀₀ value of the pathogenic bacteria.

$$Co - aggregation = [ 1 - \frac{Am}{( A 1 + A 2 ) / 2} ] \times 100 \% \qquad ( 3 )$$

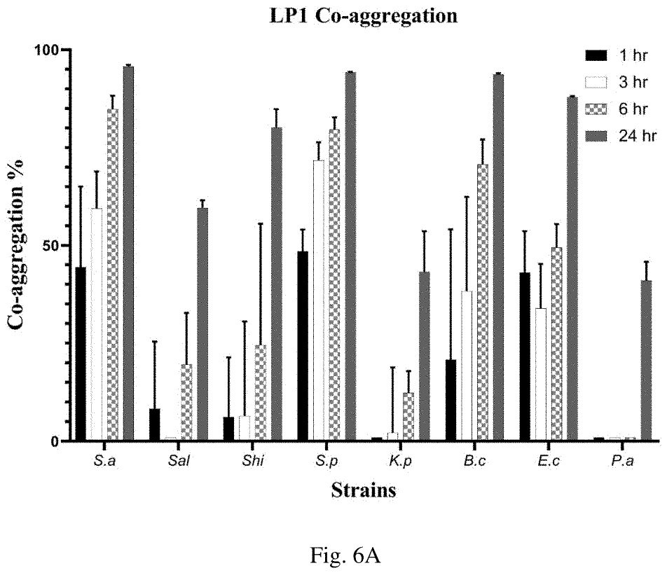

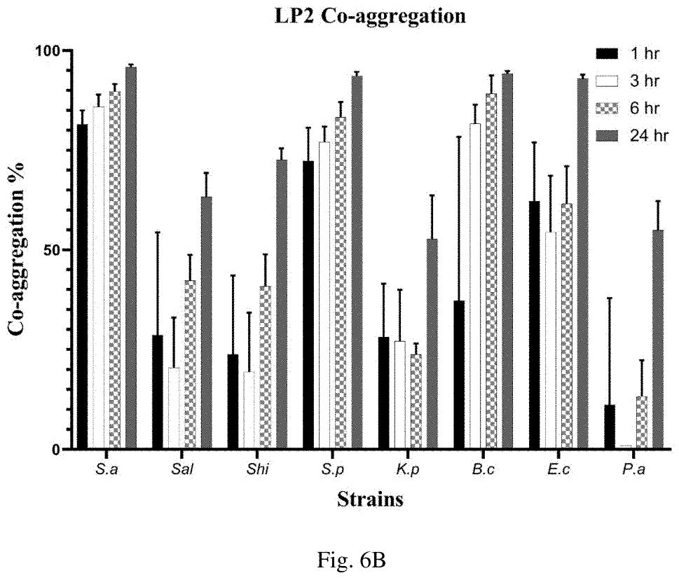

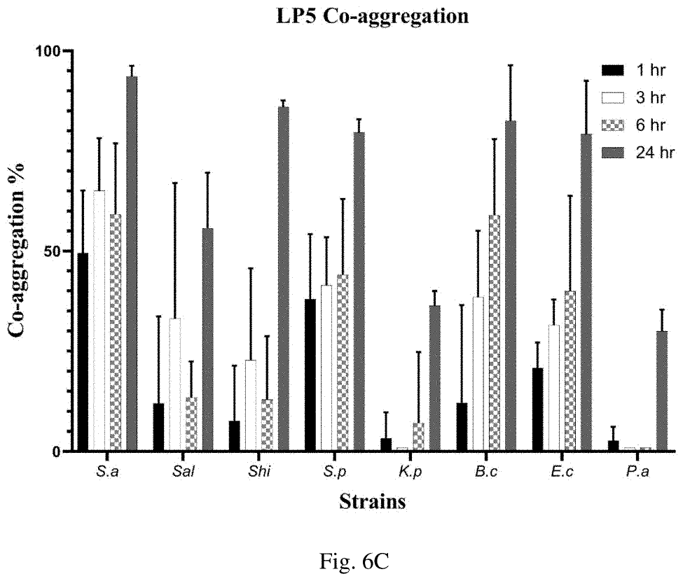

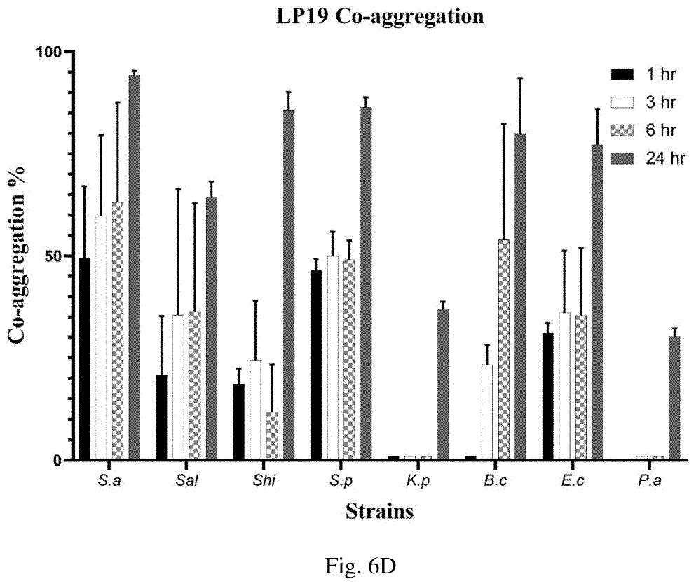

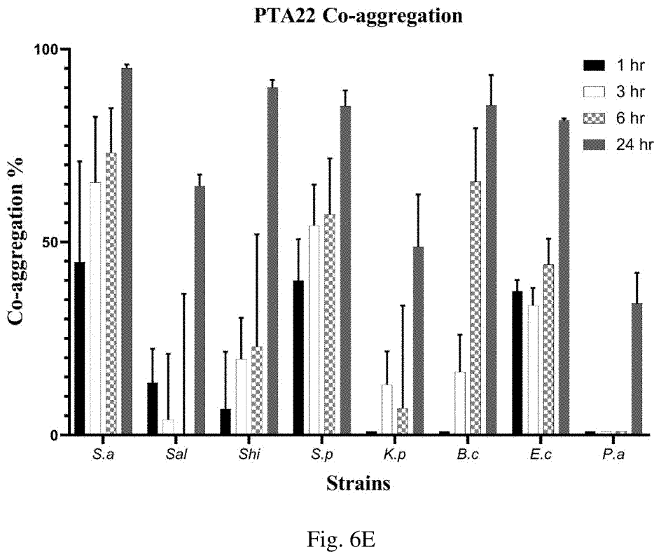

FIGS. 6A-6E shows the obtained results of the co-aggregation test of Colonies LP1, LP2, LP5, LP19, and PTA22, respectively. In FIGS. 6A-6E, the abbreviations of the pathogenic bacteria are shown in Table 11 below. From the results, it can be known that Lactiplantibacillus Plantarum LP1, LP2, LP5, LP19, and PTA22 had higher co-aggregation rate with Staphylococcus aureus, Streptococcus pneumoniae, Bacillus cereus, and Escherichia coli, and PTA22 had the highest co-aggregation rate. Hence, Colonies LP1, LP2, LP5, LP19, and PTA22 can effectively inhibit the growth of Staphylococcus aureus, Streptococcus pneumoniae, Bacillus cereus, and Escherichia coli.

TABLE 11
The abbreviations of the pathogenic bacteria.

| pathogenic bacteria | abbreviation | Gram stain |
|---|---|---|
| Staphylococcus aureus | S. a | + |
| Salmonella enterica | Sal | − |
| Shigella sonnei | Shi | − |
| Streptococcus pneumoniae | S. p | + |
| Klebsiella pneumoniae | K. p | − |
| Bacillus cereus | B. c | + |
| Escherichia coli (ETEC) | E. c | − |
| Pseudomonas aeruginosa | P. a | − |

In light of the foregoing, the bacteria of Colony PTA22 had the following characteristics. Firstly, PTA22 was more acid-tolerant but less bile salt-tolerant. In the analysis of degrading enzyme activities, the CMCase, xylanase, pectinase, and protease of PTA22 had better performance. Therefore, Lactiplantibacillus Plantarum PTA22 may be cultured in medium containing pectin and short chain fatty acids. Pectin and short chain fatty acids may be added into the rabbit feed. In the adhesion tests above, the hydrophobicity (%) and auto-aggregation (%) were not high. Hence, PTA22 may be delivered to the cecum along with the chyme and exert its CMCase and xylanase activity in the cecum or large intestine. In the analysis of the antibacterial activity, the cell-free solution of PTA22 showed antibacterial activity to many pathogenic bacteria. Comparing with Ampicillin, PTA22 was not limited by the β-lactamase to have broader antibacterial activity. Comparing with Kanamycin, PTA22 had more obvious antibacterial activity to Salmonella enterica, Shigella sonnei, Bacillus cereus, and Escherichia coli. Therefore, PTA22 can be used as probiotics for adult rabbits.

Besides, PTA22 has been deposited in NITE Patent Microorganisms Depositary (NPMD) on May 24, 2021, and the deposited number is BP-03477.

Oxalic Acid degradation Activity of PTA22

The feed that rabbits eats is too rich in calcium will cause rabbits to take in too much calcium, so that the excess calcium in the body is excreted through urine, that is calciuria. However, long-term calcariuria will burden the kidneys of rabbits, which may cause calculus and even lead to renal failure in severe cases. Thus, for rabbits, it is important whether probiotics have the ability to degrade oxalic acid. Next, the experiment tests whether PTA22 has the ability to degrade oxalic acid.

The experiment was divided to two groups. The first group: PTA22 was cultured in MRS Ox, MRS contain 10 mM/L sodium oxalate, and the second group: MRSOx with Mn²⁺, MRS contain 10 mM/L sodium oxalate and 5 mM/L MnCl₂. Oxalate concentration was using Oxalate Assay kit (Abcam, UK). Non-cultured MRSOx was used as a negative control. Besides, Oxalate degradation by intestinal lactic acid bacteria in dogs and cats (J. S. Weese et al., 2004) is as the reference of the experiment.

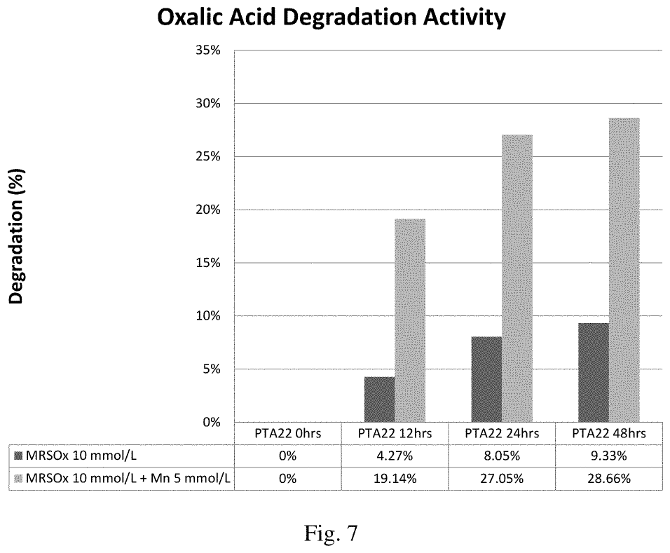

Refer to FIG. 7, which shows an oxalic acid degradation activity of PTA22 without Mn²⁺ and with Mn²⁺. Without Mn²⁺, PTA22 has the activity of degrading oxalic acid, and a degradation rate of oxalic acid is reached 9.33% at 48 hours. Since Mn²⁺ catalyzes the oxalic acid degradation reaction, a degradation rate of oxalic acid is increased at each time point after adding Mn²⁺, especially reaching 28.66% at 48 hours.

According to the literature, Oxalate degradation by intestinal lactic acid bacteria in dogs and cats (J. S. Weese et al., 2004), the oxalic acid degradation activity of PTA22 is significantly higher than the oxalic acid degradation activity of wild type. Therefore, PTA22 has oxalic acid degradation activity.

### Stress Tolerance of PTA22—Acid Tolerance and Bile Salt Tolerance

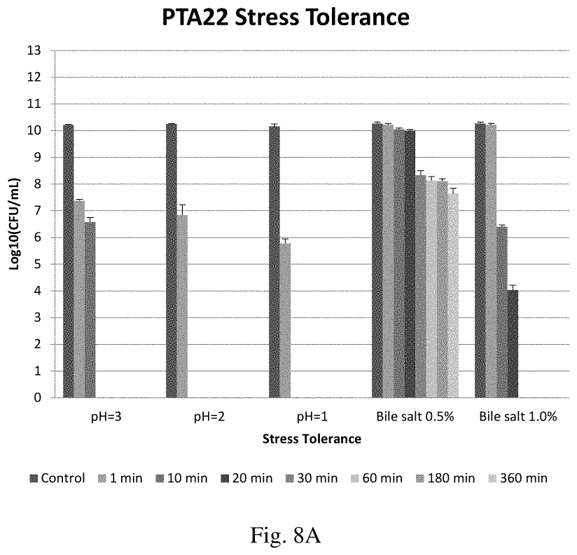

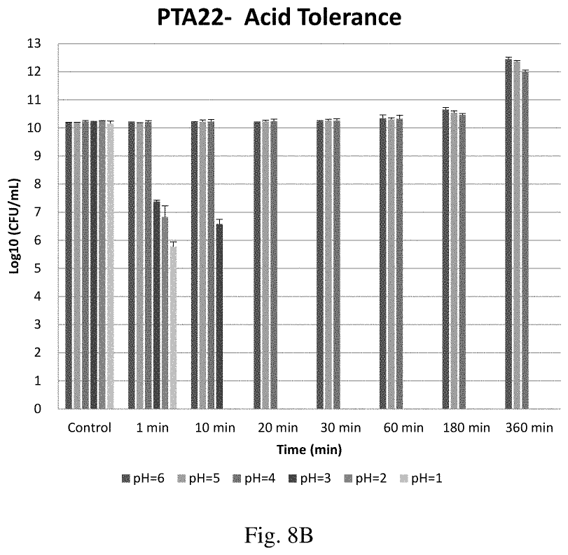

Referring to FIGS. 8A-8B, FIG. 8A shows the acid tolerance test results of PTA22 at pH 3.0, 2.0, 1.0, 0.5% bile salt and 1.0% bile salt, respectively, and FIG. 8B shows viable bacterial counts of PTA22 under different pH values at 1, 10, 20, 30, 60, 180, and 360 minutes, respectively. FIG. 8A shows that PTA22 has no acid resistance activity at pH=3.0, 2.0, 1.0, and the viable bacterial counts decrease with time. Also, and PTA22 is also not tolerant in 1.0% Bile salt. FIG. 8B shows that PTA22 can still grow sustainably in the environment of pH=6.0, 5.0, 4.0. However, PTA22 has no acid-resistant activity from pH=3.0, and the viable bacterial counts begins to decrease from pH=3.0.

As a result, since the gastric acid environment of rabbits can reach pH 1.0, and rabbits belong to hindgut fermentation, the environment in which probiotics exist in the rabbit digestive tract is very critical. However, PTA22 is not resistant to acid and bile salt, so in the manufacturing process of PTA22 in a form that can be eaten by rabbits is bound to be considerable.

For providing better antibacterial performance and help adult rabbits decompose fiber when eating foods, PTA22 is made into any form that can be ingested by rabbits. Besides, to solve the problems of the anti-acid and anti-bile salt of PTA22, PTA22 is mixed with other excipients as lyoprotectants. Test experiments with various materials as lyoprotectants are as follow.

### Single Material—a PTA22 Freezing-Dried Powder Preparation

Since the main nutrients required in the daily diet of rabbits are cellulose, proteins, carbohydrates, vitamins and minerals, the present disclosure conducts experiments on certain materials to evaluate whether the materials can be used as lyoprotectants.

Firstly, the materials are introduced. The materials for making PTA22 in a form that can be eaten by rabbits can be roughly divided into 4 categories: proteins, carbohydrates, biological materials with high biological value protein and sugar alcohols.

The proteins comprise skim milk, whey protein, soybean protein and pea protein. The carbohydrates comprise monosaccharides, disaccharides, polysaccharides and oligosaccharides. The monosaccharides comprise mannose and rhamnose. The disaccharides comprise Sucrose and Trehalose. The oligosaccharides comprise inulin, xylo-oligosaccharides and fructo-oligosaccharides.

The biological materials with high biological value protein refer to a biological material eaten by organism that can be retained in the body of organism for growth and/or maintenance, so as to reduce a feed conversion rate (FCR). The biological materials with high biological value protein comprise Moringa oleifera and Chlorella pyrenoidosa, which provides high biological value protein proteins and vitamins. Also, the biological materials were made as powder that can be easily weighed during experiments or preparing.

Further, 2.5 wt/vol % sodium glutamate, 1 wt/vol % Xanthan gum and 1 wt/vol % gum Arabic were also the test single materials in the test. Moreover, 10 wt/vol % PBS was used as a negative control.

Next, the concentrations used for each type of materials are described as follow. Actually, 5-15 wt/vol % proteins were also used in the experiment, but more than 15% proteins has the problem of oversaturation. The 5-20 wt/vol % carbohydrates were used in the experiment. However, more than 20% carbohydrates is used, as more than 20% was supersaturated.

Moreover, the biological materials have another function to assist in subsequent preparation of rabbit food as an excipient. Based on experiments, 5-10 wt/vol % biological materials have the better shaping effect.

Next, 1 mL of the single material described above mixed with pellet of PTA22. The concentration of PTA22 was measured by dissolving the PTA22 pellet in 10 mM PBS and then measuring OD₆₀₀ of the solution. If OD₆₀₀=1.00±0.02, the PTA22 solution was performed a freezing dried test. A freeze-dryer was pre-cooled at −40° C., 12 Pa for 30 minutes. The single material described above mixed with PTA22 was frozen and freeze-dried at −40° C. for 10 hours to make freeze-dried bacterial powder. Then, the single material mixed with PTA22 freeze-dried was kept at 4° C. until tested.

### Single Material—Freezing-Dried Survival Rate Test

The freeze-dried bacterial powder was re-dissolved in PBS and quantified to 1 ml to make a bacterial solution. 100 μL of the bacterial solution was serially diluted with PBS and plated on a plate. CFU/mL of the bacterial solution (as N1) was calculated with formula (4) below after incubated at 37° C. In formula (4), N₀ represents CFU/mL of the bacterial solution before freeze-dried, and N₁ represents CFU/mL of the bacterial solution after freeze-dried.

$$Freeze - dried Survival rate ( \% ) = \frac{{N}_{1}}{{N}_{0}} \times 100 \% \qquad ( 4 )$$

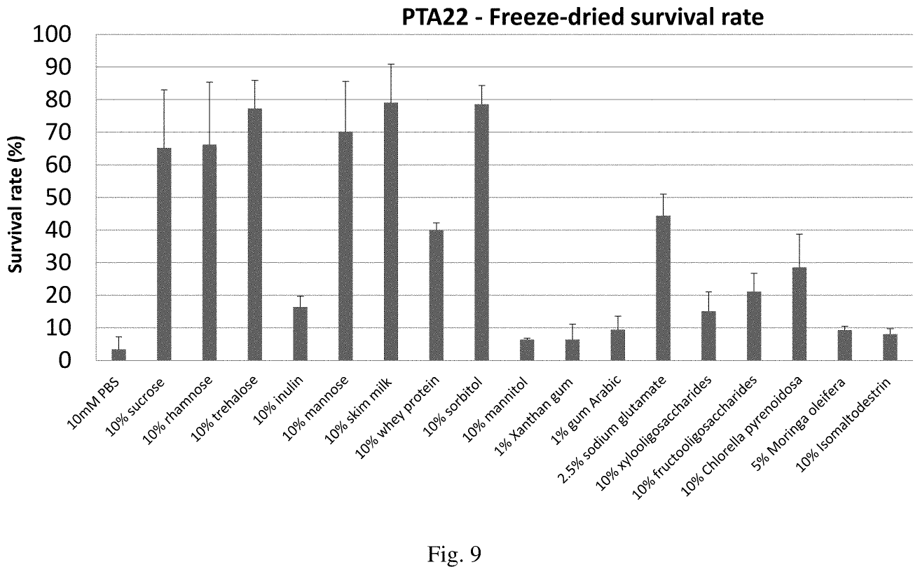

FIG. 9 shows PTA22 mixed with saccharides or proteins have better anti-freeze-dried ability. Especially, PTA22 mixed with 10 wt/vol % trahalose, 10 wt/vol % skim milk or 10 wt/vol % sorbitol has excellent freeze-dried survival rate of more than 70%. Next, PTA22 mixed with 10 wt/vol % sucrose, 10 wt/vol % rhamnose or 10 wt/vol % mannose have better freeze-dried survival rate of more than 60%.

### Single Material Test—Acid Tolerance of Freeze-Dried Bacterial Powder

The freeze-dried bacterial powders described above were added into 3 mL of MRS broth with the following condition, respectively: (1) MRS broth with pH=3.0; (2) MRS broth with pH=2.0; (3) MRS broth with pH=1.0. Next, each of 100 μL of the bacterial solutions was taken at 1, 30, 60, 180 and 360 minutes. The bacterial solutions were centrifuged at 8000 rpm for 30 seconds, and supernatants were removed. Then, pellets were washed with 100 μL of 10 mM PBS. The suspension solutions were centrifuged at 8000 rpm for 30 seconds, and supernatants were removed. 100 μL of 10 mM PBS was added to suspend the pellets, and the bacterial solutions were serially diluted with 10 mM PBS. The diluted bacterial solutions were plated on MRS agar and cultured at 37° C. for 3 days. Finally, CFU/mL of the acid-tolerance test was calculated.

Also, there was a control for the acid-tolerance test. 0.1 g of the freeze-dried bacterial powder was quantified to 1 mL with 10 mM PBS to be a bacterial solution, and 100 μL of the bacterial solution was taken for serial dilution and plated on a plate. The CFU/mL of the control for the acid-tolerance test was calculated after cultured at 37° C.

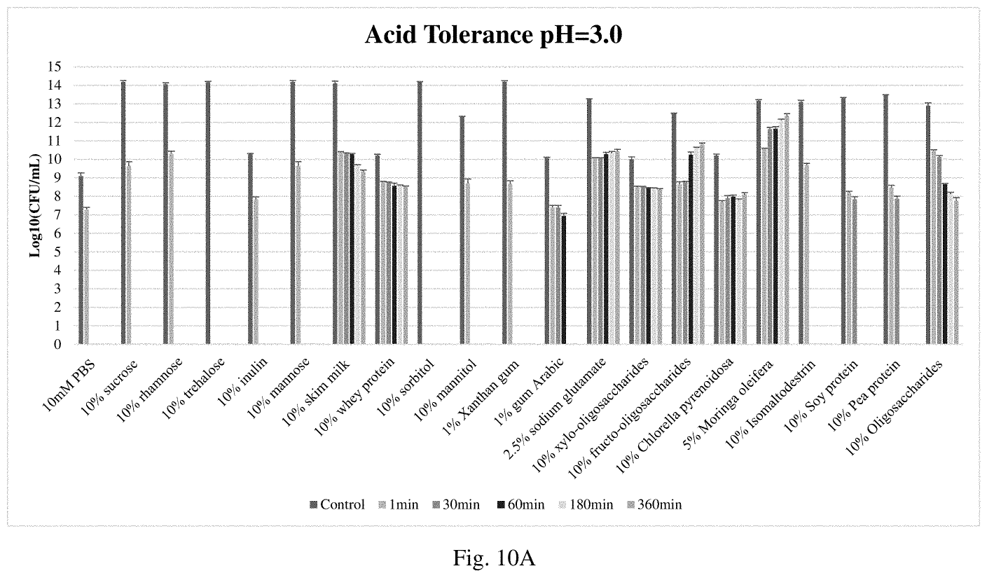

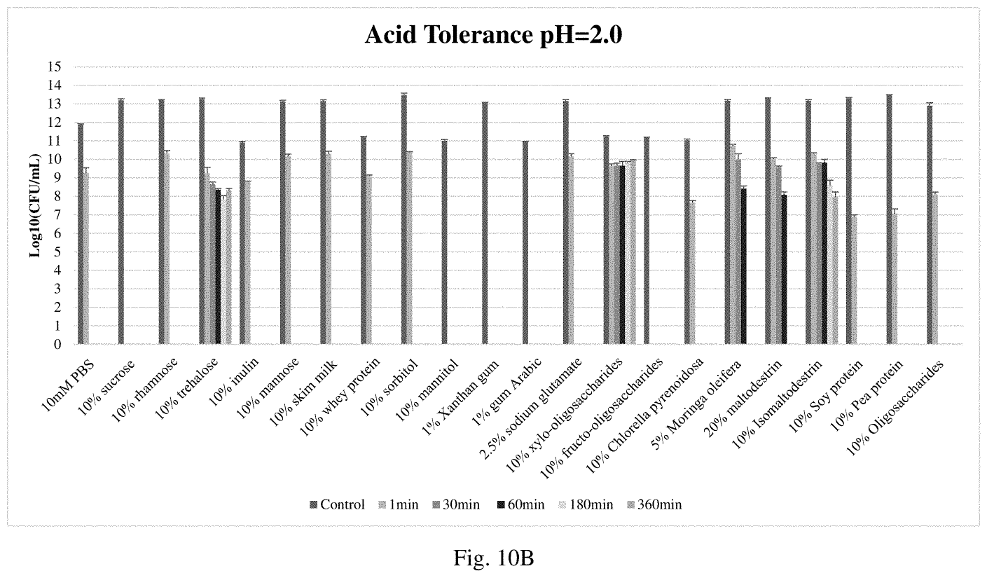

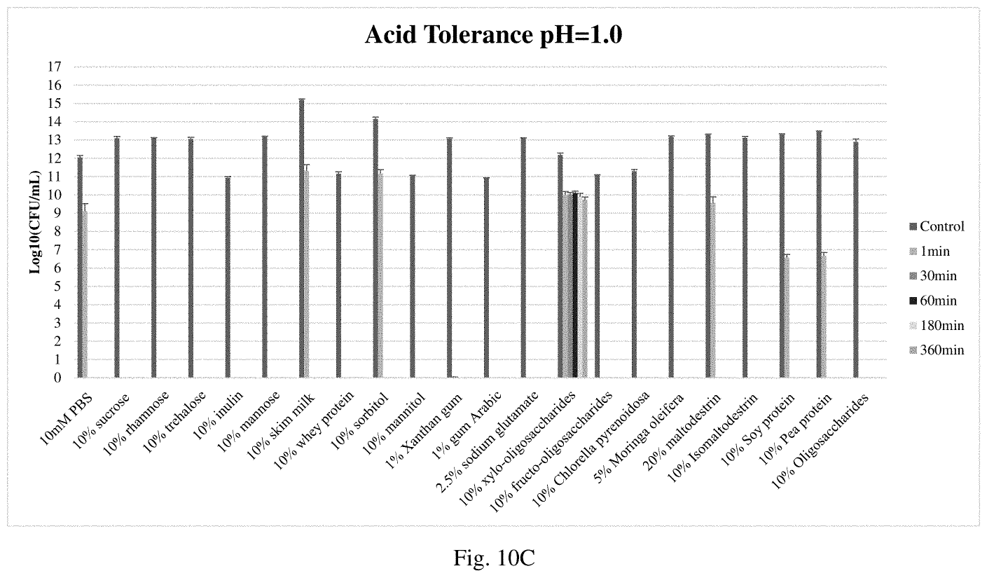

FIGS. 10A-0C show the acid tolerance test results of the freeze-dried bacterial powder at pH 3.0, 2.0, and 1.0, respectively. According to FIGS. 10A-10C, the single materials, such as 10 wt/vol % fructo-oligosaccharides, 10 wt/vol % fructo-oligosaccharide, 10 wt/vol % oligosaccharides, 2.5 wt/vol % sodium glutamate, 10 wt/vol % skim milk, 10 wt/vol % whey protein and 5 wt/vol % Moringa oleifera, can help PTA22 grow in acid environment (pH 3). 20 wt/vol % maltodextrin and 10 wt/vol % xylo-oligosaccharide, can help PTA22 still have remained viable bacteria in acid environment (pH=1.0).

Since the gastric acid of rabbits is active at pH 1.0 to 1.5, and the food passes through the stomach about 360 minutes after rabbits ingest, the data of pH 1.0 for 360 minutes in this experiment is closest to the feeding situation of rabbits. As a result, 10 wt/vol % fructo-oligosaccharide and 5 wt/vol % Moringa oleifera are very important for the subsequent development of rabbit products.

### Single Material—Bile Salt Tolerance of Freeze-Dried Bacterial Powder

The freeze-dried bacterial powders described above were added into 3 mL of MRS broth with the following condition, respectively: (1) MRS broth with 0.1% bile salt; (2) MRS broth with 0.05% bile salt. Next, each of 100 μL of the bacterial solutions was taken at 1, 30, 60, 180 and 360 minutes. The bacterial solutions were centrifuged at 8000 rpm for 30 seconds, and supernatants were removed. Then, pellets were washed with 100 μL of 10 mM PBS. The suspension solutions were centrifuged at 8000 rpm for 30 seconds, and supernatants were removed. 100 μL of 10 mM PBS was added to suspend the pellets, and the bacterial solutions were serially diluted with 10 mM PBS. The diluted bacterial solutions were plated on MRS agar and cultured at 37° C. for 3 days. Finally, CFU/mL of the bile salt tolerance test was calculated.

Also, there was a control for the bile salt tolerance test. 0.1 g of the freeze-dried bacterial powder was quantified to 1 mL with 10 mM PBS to be a bacterial solution, and 100 μL of the bacterial solution was taken for serial dilution and plated on a plate. The CFU/mL of the control for the bile salt tolerance test was calculated after cultured at 37° C.

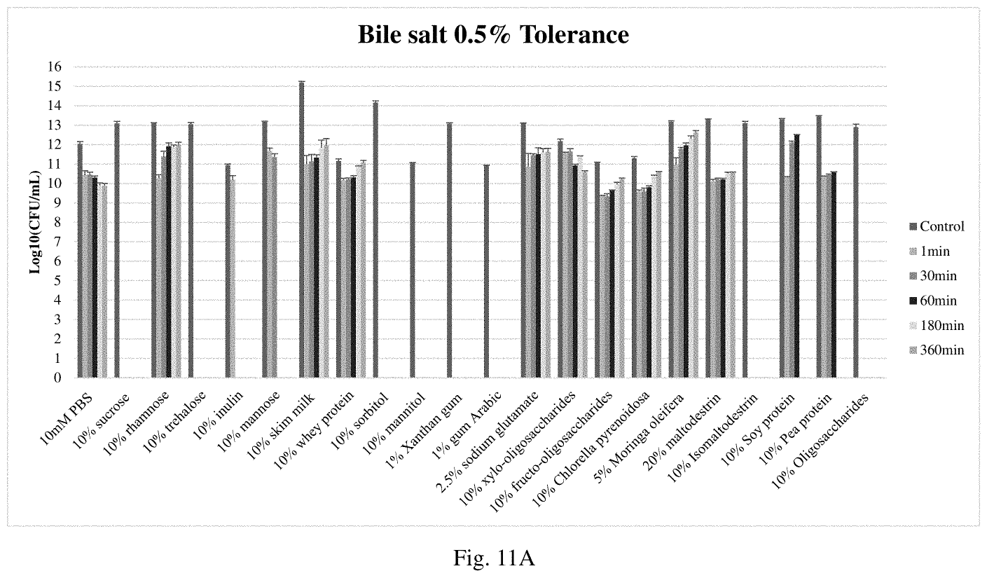

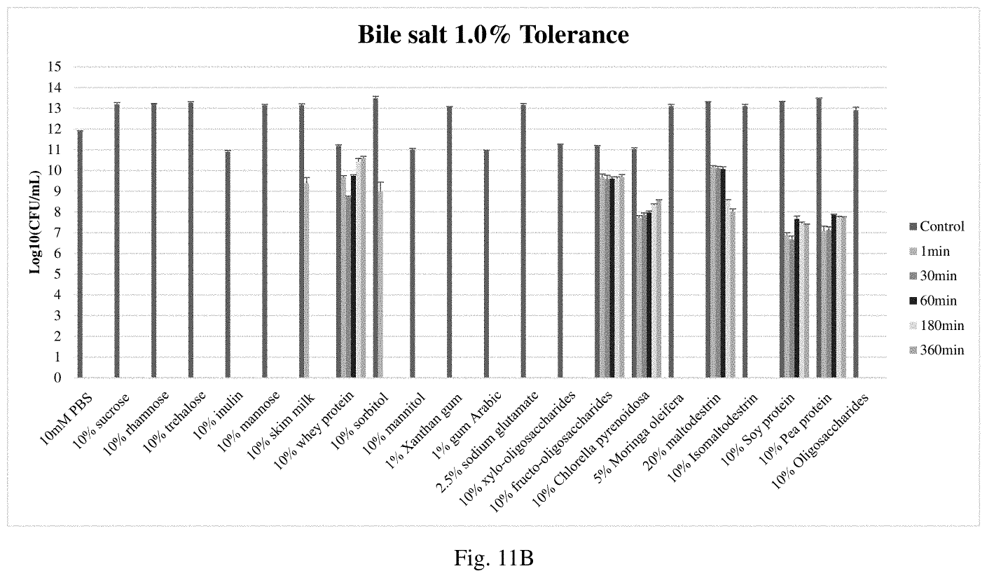

FIGS. 11A-11B show the bile salt tolerance test results of the freeze-dried bacterial powder 0.5% bile salt and 1.0% bile salt, respectively. According to FIGS. 11A-11B, the proteins, such as soybean protein and pea protein, the oligosaccharides, such as fructo-oligosaccharide and xylo-oligosaccharide, and the biological materials, such as Moringa oleifera and Chlorella pyrenoidosa, have good performance in helping PTA22 for the bile salt tolerance. It worth to mentioned that 20 wt/vol % maltodextrin also provides protection of PTA22 against bile salt tolerance.

### Compositions—Lyoprotectant for Preparing Freezing-Dried Powder of PTA22

According to the data of the freeze-dried, acid tolerance and bile salt experiments above, some materials, such as soybean protein, pea protein, fructo-oligosaccharide, xylo-oligosaccharide and Moringa oleifera, can help PTA 22 survive in acid environment. Moreover, the proteins, the oligosaccharides and the biological materials have good performance on the bile salt tolerance test. Thus, experiments of various compositions with the above-mentioned materials were further carried out. The various compositions are listed in Table 12 below.

TABLE 12
The various composition for PTA22

| | | Biological | | | |
|---|---|---|---|---|---|
| Protein | | material | | | |
| (wt/vol %) | | (wt/vol %) | Oligosaccharide (wt/vol %) | | Code |
| 5-10% Soybean | | 5-10% Moringa | 10-20% | | 5% BMX |
| protein | | oleifera | xylo-oligosaccharides | | or |
| | | | | | 10% |
| | | | 10-20% | | 5% or |
| | | | fructo-oligosaccharides | | 10% BMF |
| | | 5-10% | 10-20% | | 5% or |
| | | pyrenoidosa | xylo-oligosaccharides | | 10% BCX |
| | | Chlorella | 10-20% | | 5% or |
| | | | fructo-oligosaccharides | | 10% BCF |
| 10% Skim milk | | 5% Moringa | 10% | | SMX |
| | | oleifera | xylo-oligosaccharides | | |
| | | | 10% | | SMF |
| | | | fructo-oligosaccharides | | |
| | | 10% | 10% | | SCX |
| | | Chlorella | xylo-oligosaccharides | | |
| | | pyrenoidosa | 10% | | SCF |
| | | | fructo-oligosaccharides | | |
| 10% Whey protein | | 5% Moringa | 10% | | WMX |
| | | oleifera | xylo-oligosaccharides | | |
| | | | 10% | | WMF |
| | | | fructo-oligosaccharides | | |
| | | 10% | 10% | | WCX |
| | | Chlorella | xylo-oligosaccharides | | |
| | | pyrenoidosa | 10% | | WCF |
| | | | fructo-oligosaccharides | | |
| 5-10% Pea protein | | 5% Moringa | 10% | | 5% or |
| | | oleifera | xylo-oligosaccharides | | 10% PMX |
| | | | 10% | | 5% or |
| | | | fructo-oligosaccharides | | 10% PMF |
| | | 10% | 10% | | 5% or |
| | | Chlorella | xylo-oligosaccharides | | 10% PCX |
| | | pyrenoidosa | 10% | | 5% or |
| | | | fructo-oligosaccharides | | 10% PCF |
| 10% | 10% | 10% | 10% | 10% | 10% |
| Skim | Whey | Chlorella | fructo- | xylo- | SWCFX |
| milk | protein | pyrenoidosa | oligosaccharides | oligosaccharides | |
| 5% | 10% | 10% | 10% | 10% | 5% |
| Skim | Whey | Chlorella | fructo- | xylo- | SWCFX |
| milk | protein | pyrenoidosa | oligosaccharides | oligosaccharides | |

The descriptions for the recipes of the compositions and PTA22 are as follow. Taking SMF as an example, 10 g of skim milk, 10 g of Moringa oleifera and 10 g of fructo-oligosaccharides were taken, and ddH₂O was added to 100 mL. Finally, 1 mL of SMF was mixed with quantitated PTA22 to be freezeing-dried.

### Compositions—Freezing-Dried Test

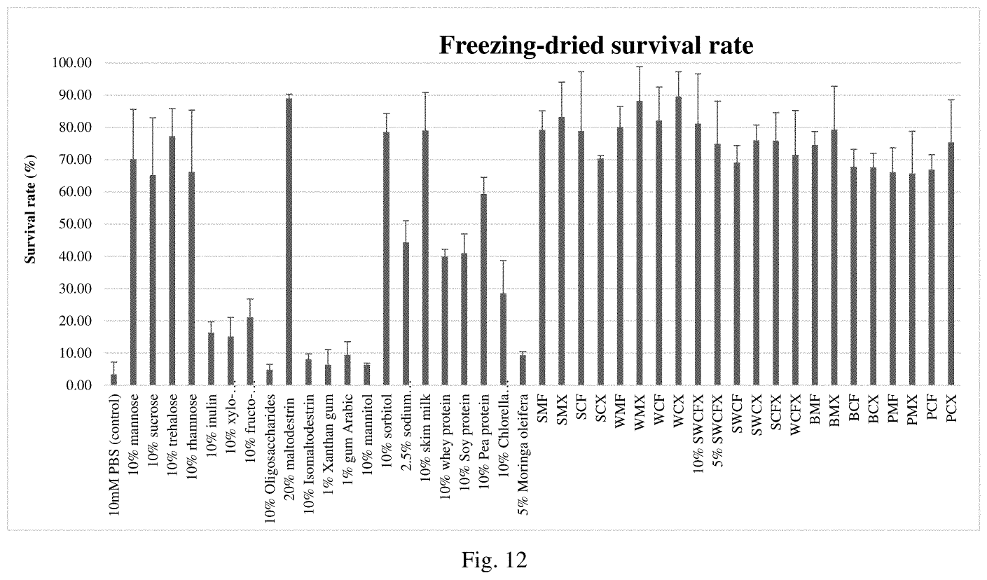

The composition described above with PTA22 were tested the freeze-dried tolerance. The experiment protocol of the freeze-dried tolerance test has described in the preceding contents, so it will not be repeated here. As the FIG. 12 showed, compared with the single lyoprotectants, the composition described above with PTA22 has relatively stable freezing-dried survival rate that more than 60%. Based on herbivore food recipes, BMF, BMX, BCF, BCX, PMF, PMX, PCF and PCX are better choices.

### Compositions—Acid-Tolerance Test

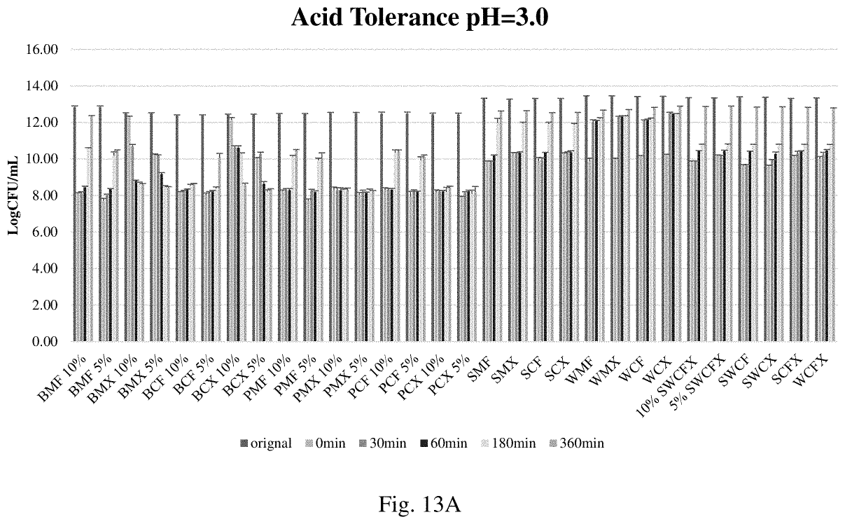

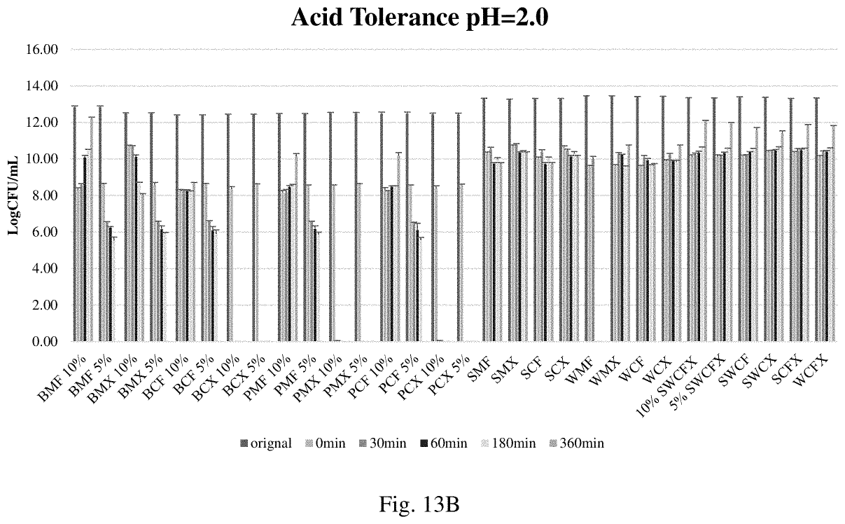

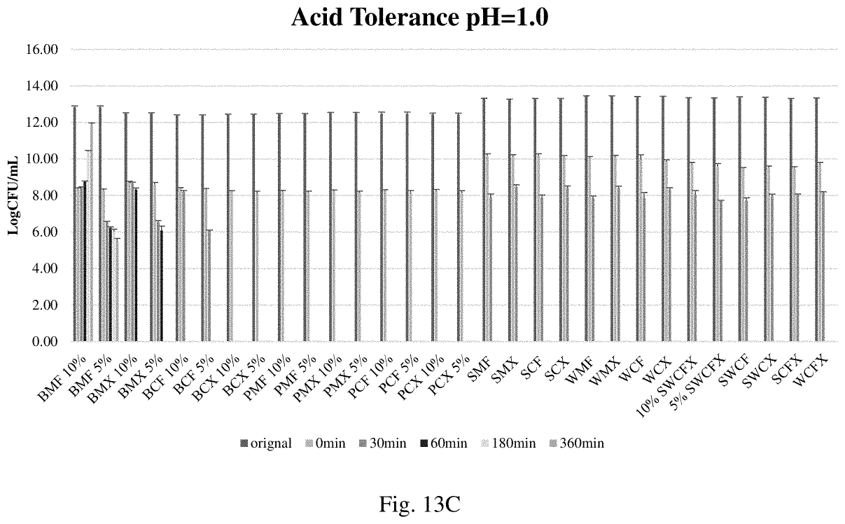

The composition described above with PTA22 were tested the acid-tolerance. The experiment protocol of the acid-tolerance test has described in the preceding contents, so it will not be repeated here. FIGS. 13A-13C show the acid tolerance test results of the composition containing PTA22 at pH 3.0, 2.0, and 1.0, respectively.

According to FIGS. 13A-13C, all the compositions have significant improvement in the acid tolerance performance of PTA22. Compared FIGS. 8A-8B with FIGS. 13A-13C, BMF can still have the viable bacteria counts that can continue to grow over time under the conditions of pH 3.0, 2.0 and 1.0. In particular to pH 1.0 for 360 minutes, BMF is most significant compared with other compositions. Thus, BMF has the best effect on helping PTA22 grow continuously in the acid environment.

### Compositions—Bile Salt Tolerance Test

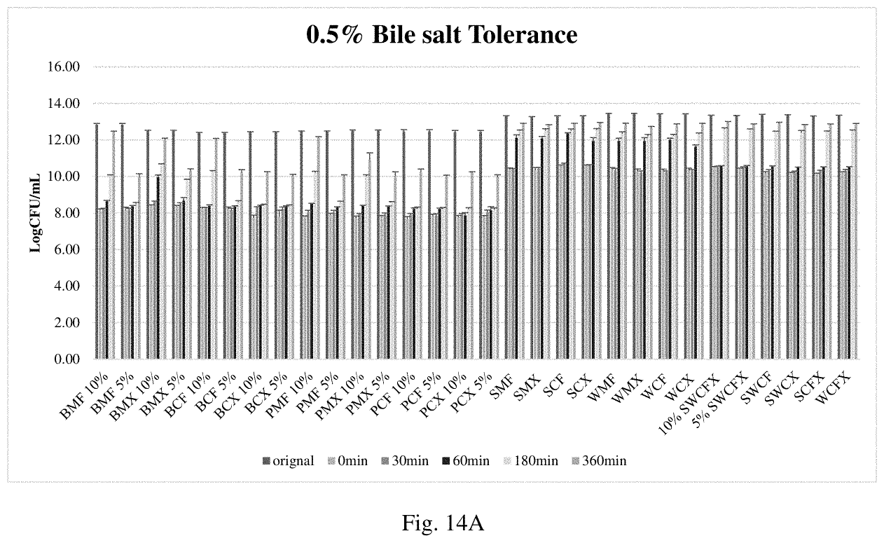

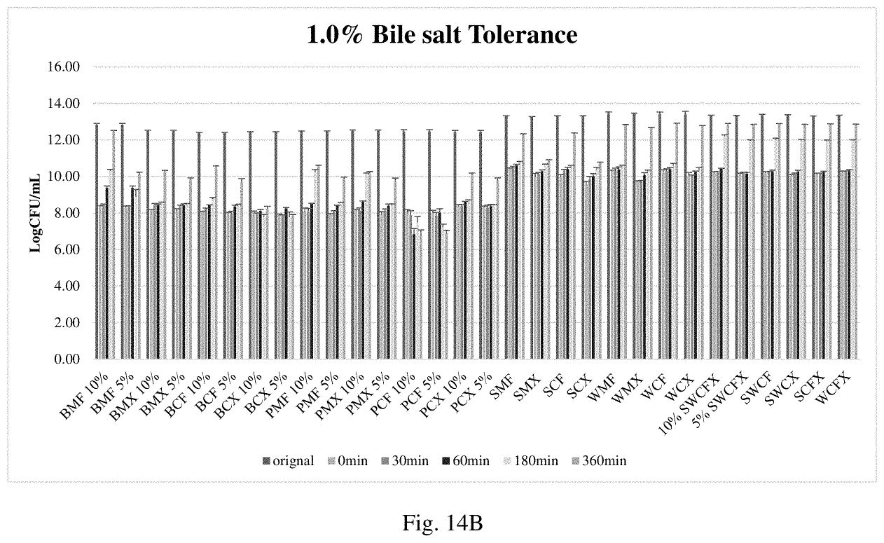

The composition described above with PTA22 were tested the bile salt tolerance. The experiment protocol of the bile salt tolerance test has described in the preceding contents, so it will not be repeated here. FIGS. 14A-14B show the bile salt tolerance test results of the composition containing PTA22 with 0.5% bile salt and 1.0% bile salt, respectively.

According to FIGS. 14A-14B, in the bile salt tolerance test, all the compositions have significant viable bacterial counts and enhance the ability of PTA22 to grow under stress compared to PTA22 without any protective agent.

Compared FIG. 8A and FIGS. 14A, PTA22 cannot grow continuously in 0.5% Bile salt environment, and PTA22 can simply maintain a certain viable bacterial counts. However, referring to FIG. 8A, PTA22 mixed with the compositions, PTA22 can continue to grow in 0.5% Bile salt environment, and the viable bacterial counts is higher than the original viable bacterial counts.

Compared FIG. 8A and FIG. 14B, PTA22 cannot survival in 1.0% Bile salt environment, and no viable bacterial count of PTA22 can be measured after 30 minutes. However, referring to FIG. 14B, PTA22 mixed with the compositions, PTA22 can continue to grow in 1.0% Bile salt environment. Among them, the compositions, such as 5-10% BMF, 10 wt/vol % BMX, 10 wt/vol % BCF, 5% PMF, 5 wt/vol % PMX and 10 wt/vol % PCX are the better lyoprotectants.

Further, in addition to considering whether the lyoprotectants have the effect of increasing the anti-acid tolerance and anti-bile salt tolerance of PTA22, considering types of subsequent preparation of food for rabbits, solubility and adhesiveness need to be paid attention. After tested, the compositions of 5% protein (Skim milk, whey protein, soybean protein or pea protein), the biological materials (10% Chlorella pyrenoidosa or 5% Moringa oleifera), and 5% oligosaccharides (fructo-oligosaccharides or xylo-oligosaccharides) were soluble and had good fluidity, but have poor adhesion and were easily to disintegrate. Hence, even though the compositions with 5% protein and 5% oligosaccharides help PTA22 with good acid-tolerance, the compositions with 5% protein and 5% oligosaccharides are not suitable for preparing food of rabbits.

The compositions of 10 wt/vol % protein (Skim milk, whey protein, soybean protein or pea protein), the biological materials (10 wt/vol % Chlorella pyrenoidosa or wt/vol 5% Moringa oleifera), and 10 wt/vol % oligosaccharides (fructo-oligosaccharides or xylo-oligosaccharides) were soluble and had good fluidity, but had good adhesion and were not easily to disintegrate. As a result, the compositions with 10% protein powder and 10% oligosaccharides are suitable for preparing food of rabbits.

### Another Compositions—Stress Tolerance Test

It is worth to mention that, as shown in FIGS. 12-14B, the single materials, maltodextrin and soy protein, has a significant improvement in the ability of freeze-dried tolerance, acid-tolerance and bile salt tolerance. Thus, the present disclosure further provides a composition that comprises 20% maltodextrin, 10% soy protein and PTA22.

Next, the composition comprising 20% maltodextrin, 10% soy protein and PTA22 were performed the acid tolerance test and the bile salt tolerance test. The protocols of the acid tolerance test and the bile salt tolerance were the same as the mentioned above, so it will not be repeated here.

To simulate an environment of probiotics particle processes, a heat tolerance test was performed as follow. 0.05 g of the freeze-dried bacterial powder was tested a heat-tolerance survival rate under the following conditions: (1) 0.05 g of the freeze-dried bacterial powder was tested at 60° C. for 30 minutes; (2) 0.05 g of the freeze-dried bacterial powder was dissolved in 200 μL of 10 mM PBS, and tested at 60° C. for 30 minutes. Then, the freeze-dried bacterial powder under different conditions was quantified to a volume of 1 mL with 10 mM PBS. Each of 100 μL of the bacterial solutions after heated was serially diluted with MRS broth to calculate CFU/mL (N₄).

As well, there was control for the heat tolerance test, and the control was made as follow. 0.05 g of the freeze-dried bacterial powder was quantified to 1 mL with 10 mM PBS to be a bacterial solution, and 100 μL of the bacterial solution was taken for serial dilution and plated on a plate. The number of original bacteria CFU/mL (as N₃) was calculated after the plate cultured at 37° C.

The heat-tolerance survival rate was calculated with formula (5). In the formula (5), N₃ represents CFU/mL of the bacterial solution at 37° C. without heating, and N₄ represents CFU/mL of the bacterial solution at 60° C.

$$Heat - tolerance Survival rate ( \% ) = \frac{{N}_{4}}{{N}_{3}} \times 100 \% \qquad ( 5 )$$

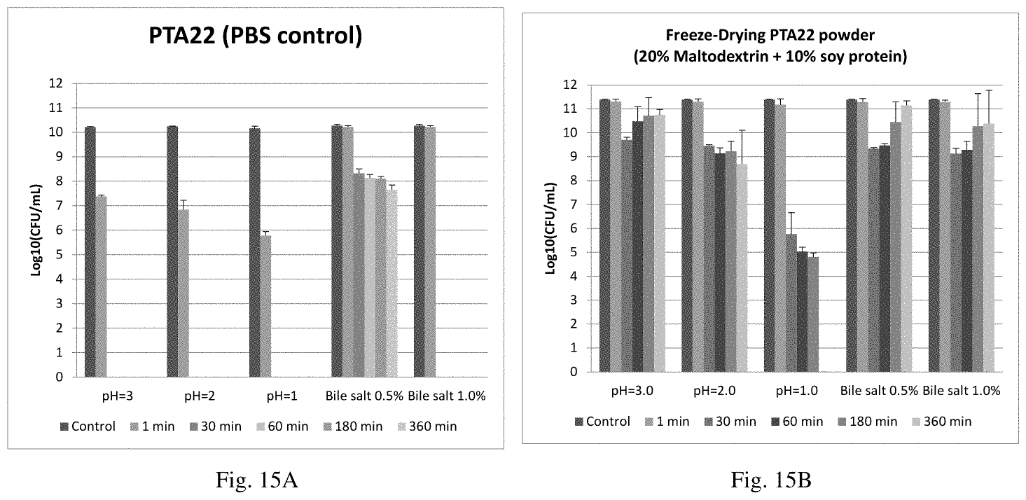

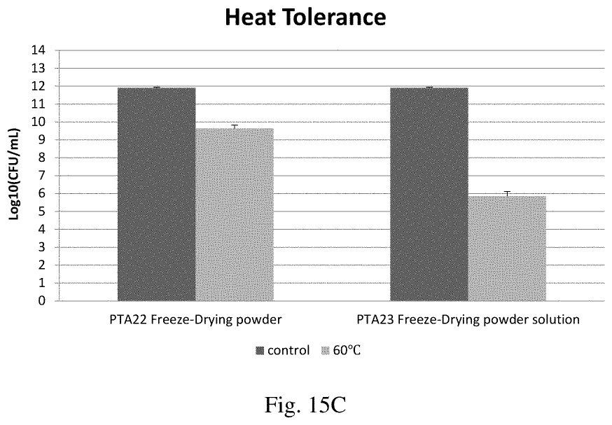

FIGS. 15A-15B show the acid tolerance test and bile salt tolerance results of a PBS control and the composition, respectively. FIG. 15C shows the heat tolerance test results of the PBS control and the composition.

As shown in FIGS. 15A-15B, compared to the PBS control, the composition comprising 20 wt/vol % maltodextrin, 10% soy protein and PTA22 has better acid-tolerance and bile salt-tolerance abilities. Furthermore, as shown in FIG. 15C, under the condition of 60° C. for 30 minutes, the water content of 0.05 g freeze-dried bacterial powder determines the survival rate in a hot environment. The survival rate of the freeze-dried bacterial powder is higher than the bacterial solution.

### Food Preparation

One of the purposes of the present disclosure is to provide food for rabbit, such as grass cake, tablets, biscuits or pellets containing probiotics, so that rabbits can obtain appropriate probiotics for digestion when eating. Hence, the next step is to mix excipient with the specific composition to obtain the food for rabbits which may be properly produced and stored.

Firstly, a preparation protocol of the food containing PTA22 was as follow. PTA22 was incubated with 3 mL of MRS broth at 37° C. for 12 hours. 100 μL of the cultured bacterial solution was subcultured in 50 mL of MRS broth at 37° C. for 12 hours for amplifying the bacterial. Next, the subcultured bacterial solution was centrifuged at 5000 rpm for 5 minutes, and supernatant was removed. A pellet were suspended with 10 mM PBS, and the suspended bacterial solution was quantified to OD₆₀₀=1.00±0.02. 1 mL of the quantified bacterial solution was taken into a 15-mL centrifuged tube. The quantified bacterial solution was centrifuged at 5000 rpm for 5 minutes, and supernatant was removed. The transferred and centrifuged step was repeated 4 times in the same 15-mL centrifuged tube to obtain 4 times the amount of the pellet. The pellet was suspended with each of 4 mL of composition solutions, respectively. After tested, the pellet of PTA22 is (1.00±0.02)×10¹² CFU/mL.

Then, 2 g of excipient, such as grass powder, herb powder, vegetable powder, fruit powder, starch powder, soybean dreg, fiber powder, or any combinations thereof was weighed with a mold. 4 mL of the composition solution was added. The composition solution with PTA22 was mixed well and compacted into grass cakes, tablets, biscuits or pellets. The grass cake was frozen at −20° C. overnight to shape. Finally, the grass cakes, tablets, biscuits or food pellets were freeze-dried with the freeze-dryer under 12 PA, at −40° C. for 10 hours. The grass cakes, tablets, biscuits or food pellets were placed in a bag and disposed in a dry box.

By the way, the grass cakes, tablets, biscuits or food pellets were with the composition added without PTA22 were prepared as controls.

TABLE 12
the contents of per grass cake, tablet, biscuit or food pellet

| | | | Biological material | | Oligosaccharide | |
|---|---|---|---|---|---|---|
| Unit: g | Excipient | Protein | Moringa oleifera | Chlorella pyrenoidosa | Xylo-oligosaccharides | Fructo-oligosaccharides |
| SMF | 2 | 0.02-0.04 | 0.01-0.02 | | | 0.02-0.04 |
| SMX | 2 | 0.02-0.04 | 0.01-0.02 | | 0.02-0.04 | |
| SCF | 2 | 0.02-0.04 | | 0.01-0.02 | | 0.02-0.04 |
| SCX | 2 | 0.02-0.04 | | 0.01-0.02 | 0.02-0.04 | |
| WMF | 2 | 0.02-0.04 | 0.01-0.02 | | | 0.02-0.04 |
| WMX | 2 | 0.02-0.04 | 0.01-0.02 | | 0.02-0.04 | |
| WCF | 2 | 0.02-0.04 | | 0.01-0.02 | | 0.02-0.04 |
| WCX | 2 | 0.02-0.04 | | 0.01-0.02 | 0.02-0.04 | |

As shown above Table 12, the nutritional composition with a weight proportion of the protein, the biological material, the oligosaccharide and the excipient is 2-4:1-2:2-4:100, and the nutritional composition contains (1.00±0.02)×10¹² CFU/mL of PTA22.

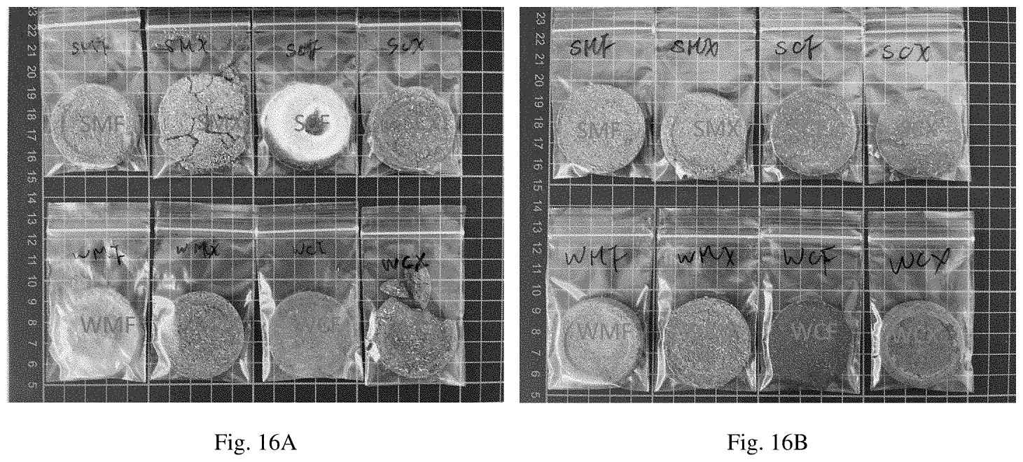

FIGS. 16A-16B show the grass cakes without PTA22 and the grass cakes with PTA22, respectively. As FIGS. 16A-16B shown, the nutritional composition added makes the shape of the food better, but the preparation is only made by freeze-dried, which is prone to mold contamination. The nutritional composition containing PTA22 significantly improve the shaping ability of the food of rabbits. As well, the grass cake containing PTA22 also reduced the occurrence of exogenous mold contamination during freeze-dried.

What is claimed is:

1. A probiotic of Lactiplantibacillus Plantarum (denoted as probiotic PTA22 below) from rabbits,
wherein the 16S rRNA gene sequence of the probiotic PTA22 is SEQ ID No: 3, the deposited number is BP-03477 in NITE Patent Microorganisms Depositary (NPMD), and
wherein the probiotic PTA22 has oxalic acid degradation activity, carboxymethyl cellulose digestion activity, pectinase digestion activity, xylanase digestion activity, and protease digestion activity,
wherein the probiotic PTA22 can inhibit the growth of a pathogenic bacterium comprising at least one of Bacillus Cereus, Staphylococcus Aureus, Klebsiella Pneumoniae, and Salmonella Enterica, Shigella Sonnei, Streptococcus Pneumoniae, Pseudomonas Aeruginosa, and E. coli (ETEC), and
wherein the probiotic PTA22 is resistant to an antibiotic comprising at least one of Aminoglycosides antibiotics, Sulfonamide antibiotics, Quinolone antibiotics, and the derivatives thereof.

2. The probiotic of claim 1, wherein the Quinolone antibiotics comprise ciprofloxacin, the Aminoglycosides antibiotics comprise kanamycin and vancomycin, and the Sulfonamide antibiotics comprise sulfamethoxazole.

3. A nutritional composition for preparing food of rabbits, comprising:
a probiotic mixture containing the probiotic PTA22 of claim 1, a postbiotic thereof, or a combination thereof;
an aqueous suspension solution of a biological material with high biological value protein;
an aqueous solution of an oligosaccharide; and
an excipient.

4. The nutritional composition for preparing food of rabbits of claim 3, further comprising an aqueous solution of a protein.

5. The nutritional composition for preparing food of rabbits of claim 4, wherein a weight proportion of the protein, the biological material, the oligosaccharide and the excipient is 2-4:1-2:2-4:100, and wherein the probiotic mixture containing (1.00±0.02)×10¹² CFU/mL of the probiotic PTA22.

6. The nutritional composition for preparing food of rabbits of claim 3, wherein the aqueous suspension solution of the biological material is selected from the group consisting of 5-10 wt/vol % Moringa oleifera and 5-10 wt/vol % Chlorella pyrenoidosa.

7. The nutritional composition for preparing food of rabbits of claim 3, wherein the aqueous solution of the oligosaccharide is selected from the group consisting of 10-20 wt/vol % xylooligosaccharide and 10-20 wt/vol % fructo-oligosaccharide.

8. The nutritional composition for preparing food of rabbits of claim 4, wherein the aqueous solution of the protein is selected from the group consisting of 5-10 wt/vol % skim milk and 5-10 wt/vol % whey protein.

9. The nutritional composition for preparing food of rabbits of claim 4, wherein the excipient is a grass powder, herb powder, vegetable powder, fruit powder, starch powder, soybean dreg, fiber powder, or any combinations thereof.

10. The nutritional composition for preparing food of rabbits of claim 9, wherein types of the food comprise grass cakes, tablets, biscuits or food pellets.

11. A composition for rabbits to degrade oxalic acid, comprising:
the probiotic PTA22 of claim 1, a postbiotic thereof, or a combination thereof in an effective amount; and
a component comprises a biological material with high biological value protein;
an aqueous solution of an oligosaccharide; and
an excipient.

12. The composition for rabbits of claim 11, wherein the biological material is selected from the group consisting of 5-10 wt/vol % Moringa oleifera and 5-10 wt/vol % Chlorella pyrenoidosa.

13. The composition for rabbits of claim 11, wherein the oligosaccharide is selected from the group consisting of 10-20 wt/vol % xylooligosaccharide and 10-20 wt/vol % fructo-oligosaccharide.

14. The composition for rabbits of claim 11, wherein the excipient is a grass powder, herb powder, vegetable powder, fruit powder, starch powder, soybean dreg, fiber powder, or any combinations thereof.


SEQUENCE LISTING (5 sequences)

SEQ ID NO: 1 - DNA, 1164 bp, Lactiplantibacillus Plantarum
  source 1..1164: mol_type = genomic DNA
tacgggaggcagcagagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccagaggaaggtggggatgacgtcaaatcatcatgccccttatgacctggctacacacgtgctacaatggatggtacaacgagttgcgaactcgcgagagtaagctaatctcttaaagccattctcagttcggattgtaggctgcaactcgcctacatgaagtcgaaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggta

SEQ ID NO: 2 - DNA, 1166 bp, Lactiplantibacillus Plantarum
  source 1..1166: mol_type = genomic DNA
tacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtcgtaacaaggta

SEQ ID NO: 3 - DNA, 1166 bp, Lactiplantibacillus Plantarum; identical to SEQ ID NO: 2
  source 1..1166: mol_type = genomic DNA

SEQ ID NO: 4 - DNA, 1118 bp, Paracoccus sp.
  source 1..1118: mol_type = genomic DNA
tacgggaggcagcagtggggaatcttagacaatgggggcaaccctgatctagccatgccgcgtgagtgatgaaggccttagggttgtaaagctctttcagctgggaagataatgacggtaccagcagaagaagccccggctaactccgtgccagcagccgcggtaatacggagggggctagcgttgttcggaattactgggcgtaaagcgcacgtaggcggactggaaagttgggggtgaaatcccggggctcaacctcggaactgccttcaaaactatcagtctggagttcgagagaggtgagtggaattccgagtgtagaggtgaaattcgtagatattcggaggaacaccagtggcgaaggcggctcactggctcgatactgacgctgaggtgcgaaagcgtggggagcaaacaggattagataccctggtagtccacgccgtaaacgatgaatgccagacgtcgggcagcatgctgttcggtgtcacacctaacggattaagcattccgcctggggagtacggtcgcaagattaaaactcaaaggaattgacgggggcccacacaagcggtggagcatgtggtttaattcgaagcaacgcgcagaaccttaccaacccttgacattgcaggacatcccgagagatcgggctttcacttcggtgacctgcggacaggtgctgcatggctgtcgtcagctcgtgtcgtgagatgttcggttaagtccggcaacgagcgcaacccacgtccccagttgccagcattcagttgggcactctgtggaaactgccgatgataagtcggaggaaggtgtggatgacgtcaagtcctcatggcccttacgggttgggctacacacgtgctacaatggtggtgacagtgggttaatccccaaaagccatctcagttcggattggggtctgcaactcgaccccatgaagttggaatcgctagtaatcgcggaacagcatgccgcggtgaatacgttcccgggccttgtacacaccgcccgtcacaccatgggagttggttctacccgacgacgctgcgctaacccttacgggaggcaggcggccacggtaggatcagcgactggggtgaagtcgtaacaaggta

SEQ ID NO: 5 - DNA, 1182 bp, Staphylococcus lentus
  source 1..1182: mol_type = genomic DNA
tacgggaggcagcagtagggaatcttccgcaatgggcgaaagcctgacggagcaacgccgcgtgagtgatgaaggtcttaggatcgtaaaactctgttgttagggaagaacaaatttgttagtaactgaacaagtcttgacggtacctaaccagaaagccacggctaactacgtgccagcagccgcggtaatacgtaggtggcaagcgttatccggaattattgggcgtaaagcgcgcgtaggcggtttcttaagtctgatgtgaaagcccacggctcaaccgtggagggtccgtggagggtcattggaaactggggaacttgagtgcagaagaggagagtggaattccatgtgtagcggtgaaatgcgcagagatatggaggaacaccagtggcgaaggcggctctctggtctgtaactgacgctgatgtgcgaaagcgtggggatcaaacaggattagataccctggtagtccacgccgtaaacgatgagtgctaagtgttagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctttgaccgctctagagatagagttttccccttcgggggacaaagtgacaggtggtgcatggttgtcgtcagctcgtgtcgtgagatgttgggttaagtcccgcaacgagcgcaacccttaagcttagttgccatcattaagttgggcactctaggttgactgccggtgacaaaccggaggaaggtggggatgacgtcaaatcatcatgccccttatgatttgggctacacacgtgctacaatggataatacaaagggcagcgaacccgcgaggtcaagcaaatcccataaaattattctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttattaggagctagccgtcgaaggtgggacagatgattggggtgaagtcgtaacaaggta